(19) United States
(12) Patent Application Publication
(10) Pub. No.: US 2024/0009025 A1
(43) Pub. Date: Jan. 11, 2024

(54) THERMAL CONTROL SYSTEMS WITH DYNAMIC CONTROL ALGORITHMS

(71) Applicant: Stryker Corporation, Kalamazoo, MI (US)

(72) Inventors: Marco Constant, Portage, MI (US); Anish Paul, Portage, MI (US); Marko N. Kostic, Oshawa, Ontario (CA); Sujay Sukumaran, Portage, MI (US); Krishna Sandeep Bhimavarapu, Kalamazoo, MI (US); Alexey Titov, Redmond, WA (US)

(21) Appl. No.: 18/028,314

(22) PCT Filed: Dec. 6, 2021
(86) PCT No.: PCT/US2021/061947

§ 371 Date: Mar. 24, 2023

Related U.S. Application Data
(60) Provisional application No. 63/122,165, filed on Dec. 7, 2020.

(51) Int. Cl. A61F 7/00; G05B 13/02

(52) U.S. Cl. CPC A61F 7/0085; G05B 13/027; A61F 2007/0054; A61F 2007/0086; A61F 2007/0096; A61F 2007/0093

(57) ABSTRACT

A thermal control system for controlling a patient's temperature includes a thermal control unit and an off-board computing device. The thermal control unit includes a fluid inlet, a fluid outlet, a pump, a heat exchanger, a display, one or more sensors, a transceiver, and a controller. The thermal control system employs one or more machine learning techniques to perform one or more of the following: automatically implement one or more user-preferred settings, automatically predict the occurrence of one or more events based on analyses of prior events, and/or automatically improve one or more algorithms based on analyses of additional sensor data. The machine learning techniques may be implemented onboard the thermal control unit and/or may be implemented at a remote computing device (e.g. a server) that collates and analyzes data from multiple thermal control units, and then sends the results of the analyses back to the thermal control units.

### BACKGROUND

The present disclosure relates to a thermal control system for controlling the temperature of circulating fluid that is delivered to one or more thermal devices positioned in contact with a patient.

Thermal control systems are known in the art for controlling the temperature of a patient by providing a thermal control unit that supplies temperature-controlled fluid to one or more thermal pads or catheters positioned in contact with a patient. The thermal control unit includes one or more heat exchangers for controlling the temperature of the fluid and a pump that pumps the temperature-controlled fluid to the pad(s) and/or catheter. After passing through the pad(s) and/or catheter, the fluid is returned to the thermal control unit where any necessary adjustments to the temperature of the returning fluid are made before being pumped back to the pad(s) and/or catheter. In some instances, the temperature of the fluid is controlled to a static target temperature, while in other instances the temperature of the fluid is varied as necessary in order to automatically effectuate a target patient temperature.

Thermal control units typically include a control panel adapted to allow the user to input information for using the thermal control unit, as well as for displaying information useful to the user of the thermal control unit. The control panel also enables the user to execute one or more functions of the patient support apparatus, such as, but not limited to, inputting a target patient temperature; choosing a cooling rate; choosing a warming rate; defining a cooling, warming, and/or hold time; determining which alarms to implement; selecting alarm characteristics; controlling what information is displayed, recorded, and/or transmitted off-board the thermal control unit; choosing what sensor inputs are to be used during the thermal therapy session, etc. Some of these functions are carried out in different manners, depending upon one or more settings that that can be selected by the user. Some of these functions may also be carried out by one or more algorithms that rely on data from one or more sensors, and the set of sensor(s) used by the algorithm does not change over time.

### SUMMARY

A thermal control system according to one or more embodiments of the present invention provides thermal control units that are adapted to use machine learning to improve their operation over time. Such improvements may relate to automatically selecting one or more user-preferred settings after gathering data from previous selections by users. Additionally or alternatively, such improvements may relate to improving the prediction of one or more events and/or improving one or more algorithms by using additional sensor data that is determined to have a predictable influence on the function(s) carried out by the algorithm. In some embodiments, the predicted event and/or improved algorithm relate to a patient starting to shiver, a patient's temperature overshooting a target temperature for the patient, an alarm being issued, and/or a user making a selection of one or more user-preferred settings associated with a function of the thermal control unit. Further, in some embodiments, the improved algorithm utilizes sensor data from sensors that weren't used in the previous algorithm, but which were determined through the machine learning process to provide useful information.

A thermal control unit according to one embodiment of the present disclosure includes a fluid outlet, a fluid inlet, a circulation channel, a pump, a heat exchanger, a fluid temperature sensor, a patient temperature sensor port, a display, a control, a transceiver, and a controller. The circulation channel couples the fluid inlet to the fluid outlet. The pump circulates fluid through the circulation channel from the fluid inlet to the fluid outlet. The heat exchanger is adapted to add or remove heat from the fluid circulating in the circulation channel. The fluid temperature sensor is adapted to sense a temperature of the fluid, and the patient temperature sensor port is adapted to receive patient temperature readings from a patient temperature sensor. The control is adapted to be activated by a user of the thermal control unit, and the controller is adapted to control the heat exchanger in order to control the patient's temperature. The controller is further adapted to perform a function of the thermal control unit when the control is activated by the user. The function is capable of being performed in a plurality of different manners based upon a setting selectable by the user, and the controller is further adapted to record over time setting data indicating the setting selected by the user when the function is performed. The transceiver is adapted to transmit the setting data to a computing device located off-board the thermal control unit, and the computing device is adapted to analyze the setting data to determine a user-preferred setting when the function is performed. The controller is still further adapted to receive a message back from the computing device indicating the user-preferred setting and to automatically select the user-preferred setting when the user activates the control.

According to other aspects of the present disclosure, the function may be an implementation of a temperature alert wherein the temperature alert is issued when the patient's temperature differs from a target temperature by more than a threshold. In such embodiments, the user-preferred setting may include any one or more of the following: an audio characteristic of the temperature alert; a priority level of the temperature alert; a repetition setting for the temperature alert; a delay period between multiple temperature alerts; a pause availability for the temperature alert; a pause duration selection for the temperature alert; or a remote notification setting of the temperature alert.

In some embodiments, the function is the implementation of a flow rate alert wherein the flow rate alert is issued when a rate of flow of the fluid through the circulation channel falls below a threshold. In such embodiments, the user-preferred setting may include one or more of the following: an audio characteristic of the flow rate alert; a priority level of the flow rate alert; a repetition setting for the flow rate alert; a delay period between multiple flow rate alerts; a pause availability for the flow rate alert; a pause duration selection for the flow rate alert; or a remote notification setting of the flow rate alert.

In some embodiments, the function is the implementation of a therapy profile wherein the therapy profile dictates how the thermal control unit seeks to control the patient's temperature during the thermal therapy session. In such embodiments, the user-preferred setting may include one or more of the following: a target temperature for the patient; a duration for which the patient is to be maintained at the target temperature; a rate at which the patient's temperature is to be cooled; a rate at which the patient's temperature is to be warmed; or a target time to achieve the target temperature for the patient.

In some embodiments, the thermal control unit further includes at least one sensor and the controller is further adapted to take multiple sets of readings from that sensor and record the sets of readings. Each set of the multiple sets of readings including readings taken both before and after an occurrence of an event associated with the thermal control unit, and the controller is further adapted to transmit the sets of readings to the computing device. In such embodiments, the controller may further be adapted to receive an algorithm back from the computing device for predicting a future occurrence of the event.

In some embodiments, the thermal control unit includes a plurality of additional sensors and the controller is further adapted to take a set of readings from these plurality of additional sensors, record the set of readings, and use a first subset of the set of readings in an algorithm for performing the function of the thermal control unit. In such embodiments, the first subset excludes readings from at least one sensor in the plurality of sensors and the controller is further adapted to transmit the set of readings to the computing device.

In some embodiments, the controller is further adapted to receive an improved algorithm back from the computing device and to use the improved algorithm when performing the function. In such embodiments, the improved algorithm uses a second subset of the readings from the plurality of sensors that is different from the first subset. In some of these embodiments, the second subset of the readings does not exclude readings from the at least one sensor in the plurality of sensors. Still further, in some of these embodiments, the first subset includes at least one reading from a sensor not included in the second subset.

According to another embodiment of the present disclosure, a thermal control system is provided that includes a plurality of thermal control units and a computing device positioned remotely from the plurality of thermal control units. Each of the thermal control units is adapted to control a patient's temperature during a thermal therapy session, and each of the thermal control units includes a fluid outlet, a fluid inlet, a circulation channel, a pump, a heat exchanger, a display, a sensor, a transceiver, and a controller. The circulation channel is coupled to the fluid inlet and the fluid outlet and the pump is adapted to circulate fluid through the circulation channel from the fluid inlet to the fluid outlet. The heat exchanger is adapted to add or remove heat from the fluid circulating in the circulation channel. The controller is adapted to control the heat exchanger in order to control the patient's temperature and is also adapted to take multiple sets of readings from the sensor and record the sets of readings, wherein each set of the multiple sets of readings including readings taken both before and after an occurrence of an event associated with the thermal control unit. The computing device is in communication with the transceivers of the thermal control units. The computing device is adapted to receive the sets of readings from the thermal control units and to analyze the sets of readings to determine an algorithm for predicting a future occurrence of the event using future readings from the sensors of the thermal control units.

According to other aspects of the present disclosure, the event may be the patient shivering and the sensor may be a temperature sensor adapted to detect the patient's temperature.

In some embodiments, the sensor includes at least one additional sensor and the controller is adapted to receive an additional set of readings from the additional sensor, to record the additional set of readings, and to transmit the additional set of readings to the computing device. In such embodiments, the computing device is further adapted to analyze the additional set of readings to determine the algorithm for predicting a future occurrence of the event using future readings from both the sensor and the additional sensor of the thermal control units.

The additional sensor, in some embodiments, includes at least one of the following: a fluid temperature sensor adapted to detect a temperature of the circulating fluid; a first patient temperature sensor adapted to detect a patient's core temperature; a second patient temperature sensor adapted to detect the patient's peripheral temperature; a flow rate sensor adapted to detect a rate of flow of fluid through the fluid outlet; a clock; or a transceiver adapted to receive a message from a weight sensor positioned off-board the thermal control units.

In some embodiments, the controller is further adapted to control a temperature of the circulating fluid using a second algorithm, and the second algorithm is based at least partially on outputs from the temperature sensor.

In some embodiments, the computing device is adapted to use a neural network to generate the improved second algorithm. In such embodiments, the computing device may be adapted to use at least two of the following as inputs into the neural network: a patient weight, a patient age, a patient Body Mass Index (BMI), a time of day, an amount of time since the patient last exited from the thermal control unit, a calendar date, or what type of medication the patient has taken.

In some embodiments, the controller is further adapted to perform a function of the thermal control unit when a control is activated by a user, wherein the function is performed in a plurality of different manners based upon a setting selectable by the user. In such embodiments, the event may be the selection of the setting by the user, and the algorithm may be adapted to predict a future setting in response to the user activating the control.

According to another embodiment of the present disclosure, a thermal control system is provided that includes a plurality of thermal control units and a computing device positioned remotely from the plurality of thermal control units. Each of the thermal control units is adapted to control a patient's temperature during a thermal therapy session, and each of the thermal control units includes a fluid outlet, a fluid inlet, a circulation channel, a pump, a heat exchanger, a display, a plurality of sensors, a transceiver, and a controller. The circulation channel is coupled to the fluid inlet and the fluid outlet and the pump is adapted to circulate fluid through the circulation channel from the fluid inlet to the fluid outlet. The heat exchanger is adapted to add or remove heat from the fluid circulating in the circulation channel. The controller is adapted to control the heat exchanger in order to control the patient's temperature. The controller is also adapted to take a set of readings from the plurality of sensors and use a first subset of the set of readings in an algorithm for performing a function of the thermal control unit. The first subset excludes readings from at least one sensor in the plurality of sensors. The controller is further adapted to record the set of readings and transmit them to the computing device via the transceiver. The computing device is adapted to receive the sets of readings from the plurality of thermal control units and to determine an improved algorithm for use by each of the plurality of thermal control units when performing the function.

According to other aspects of the disclosure, the controller may further be adapted to receive the improved algorithm back from the computing device and to use the improved algorithm when performing the function. In such embodiments, the improved algorithm may use a second subset of the readings from the plurality of sensors that is different from the first subset.

In some embodiments, the second subset of the readings does not exclude readings from the at least one sensor in the plurality of sensors.

In some embodiments, the first subset includes at least one reading from a sensor not included in the second subset.

The function, in some embodiments, is cooling the patient to a target temperature and the first subset includes a patient temperature sensor adapted to measure a core temperature of the patient.

The function, in some embodiments, is detecting shivering in the patient and the first subset includes a patient temperature sensor adapted to measure a core temperature of the patient.

In some embodiments, the plurality of sensors further includes one or more of the following: a fluid temperature sensor adapted to detect a temperature of the circulating fluid; a second patient temperature sensor adapted to detect a patient's peripheral temperature; a flow rate sensor adapted to detect a rate of flow of fluid through the fluid outlet; a clock; or a transceiver adapted to receive a message from a device positioned off-board the thermal control units.

In some embodiments, the plurality of sensors includes a transceiver adapted to receive a message from a device positioned off-board the thermal control unit, and the message includes one or more of the following data items: an age of the patient, a weight of the patient, a height of the patient, or a Body Mass Index (BMI) of the patient.

In some embodiments, the computing device is adapted to use a neural network to analyze the set of readings and determine the improved algorithm. In such embodiments, the computing device may be adapted to use at least two of the following as inputs into the neural network: an age of the patient, a weight of the patient, a height of the patient, a Body Mass Index (BMI) of the patient, an ambient humidity reading, an ambient air temperature reading, a catheter liquid temperature, a room air flow reading, or a temperature of a support surface upon which the patient is positioned.

The computing device, in some embodiments, may further be adapted to use at least two of the following as additional inputs the neural network: an amount of time since the thermal therapy session began, an amount of time since a patient target temperature was set, an amount of time since the current patient temperature differed from the patient target temperature by more than a threshold; a temperature of a thermal pad fluidly coupled to the fluid inlet and fluid outlet; an incidence frequency of past patient shivering events for the healthcare facility; an incidence frequency of past shivering events for a particular department of the healthcare facility; an incidence frequency of past shivering events for a particular caregiver; an incidence frequency of past shivering events for a floor of the healthcare facility; an incidence frequency of past shivering events for a particular room in which the thermal control units are each positioned in; a presence of family members visiting the patient; or whether the thermal controls units are being used during surgery or not.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration is used in the description herein of various embodiments (e.g. first, second, third, etc.). Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a thermal control system according to one aspect of the present disclosure shown applied to a patient on a patient support apparatus;

FIG. 2 is a perspective view of a thermal control unit of the thermal control system of FIG. 1;

FIG. 3 is a block diagram of an illustrative embodiment of the thermal control system of FIG. 1;

FIG. 4 is a plan view of a control panel of the thermal control unit;

FIG. 5 is a flow diagram of an automatic setting selection algorithm that may be followed by the thermal control unit;

FIG. 6 is an example of an alarm selection screen displayable on a display of the thermal control unit;

FIG. 7 is an example of an alarm customization screen displayable on the thermal control unit;

FIG. 8 is an example of a therapy profile selection screen displayable on the thermal control unit;

FIG. 9 is an example of a therapy profile customization screen displayable on the thermal control unit;

FIG. 10 is an example of a graph customization screen displayable on the thermal control unit;

FIG. 11 is an example of a hospital location selection screen displayable on the thermal control unit;

FIG. 12 is an example of a user selection screen displayable on the thermal control unit;

FIG. 13 is a flow diagram of a future event prediction algorithm that may be followed by the thermal control unit; and

FIG. 14 is a diagram of a neural network that may be used by the thermal control unit and/or a computer device located off-board the thermal control unit in order to improve a patient cooling algorithm associated with the thermal control unit.

### DETAILED DESCRIPTION OF THE EMBODIMENTS

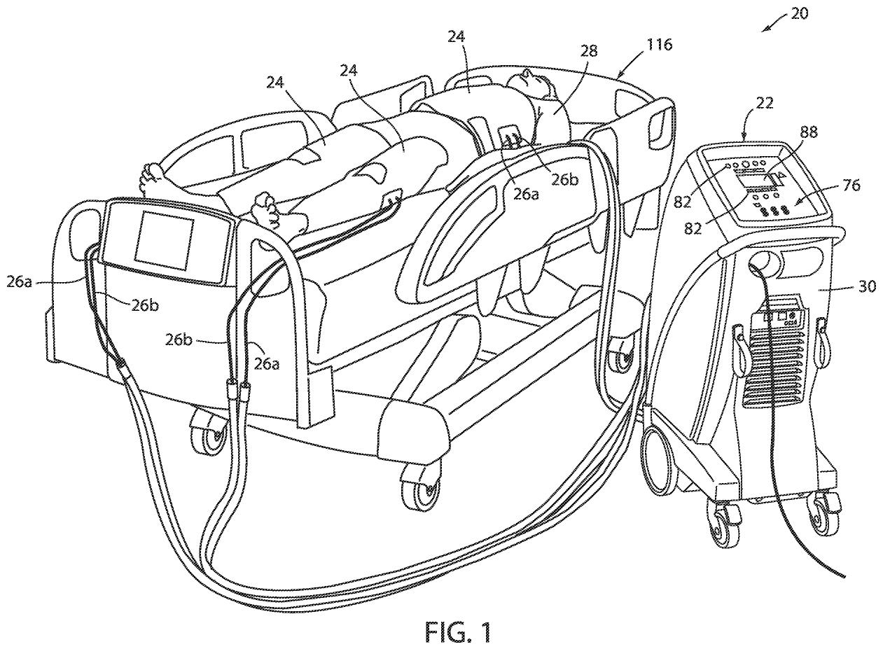

A thermal control system 20 according to one embodiment of the present disclosure is shown in FIG. 1. Thermal control system 20 is adapted to control the temperature of a patient 28, which may involve raising, lowering, and/or maintaining the patient's temperature. Thermal control system 20 includes a thermal control unit 22 coupled to one or more thermal therapy devices 24. The thermal therapy devices 24 are illustrated in FIG. 1 to be thermal pads, but it will be understood that thermal therapy devices 24 may take on other forms, such as, but not limited to, blankets, vests, patches, caps, catheters, or other structures that receive temperature-controlled fluid. For purposes of the following written description, thermal therapy devices 24 will be referred to as thermal pads 24, but it will be understood by those skilled in the art that this terminology is used merely for convenience and that the phrase “thermal pad” is intended to cover all of the different variations of thermal therapy devices 24 mentioned above (e.g. blankets, vests, patches, caps, catheters, etc.) and variations thereof.

Thermal control unit 22 is coupled to thermal pads 24 via a plurality of hoses 26. Thermal control unit 22 delivers temperature-controlled fluid (such as, but not limited to, water or a water mixture) to the thermal pads 24 via the fluid supply hoses 26a. After the temperature-controlled fluid has passed through thermal pads 24, thermal control unit 22 receives the temperature-controlled fluid back from thermal pads 24 via the return hoses 26b.

In the embodiment of thermal control system 20 shown in FIG. 1, three thermal pads 24 are used in the treatment of patient 28. A first thermal pad 24 is wrapped around a patient's torso, while second and third thermal pads 24 are wrapped, respectively, around the patient's right and left legs. Other configurations can be used and different numbers of thermal pads 24 may be used with thermal control unit 22, depending upon the number of inlet and outlet ports that are included with thermal control unit 22. By controlling the temperature of the fluid delivered to thermal pads 24 via supply hoses 26a, the temperature of the patient 28 can be controlled via the close contact of the pads 24 with the patient 28 and the resultant heat transfer therebetween.

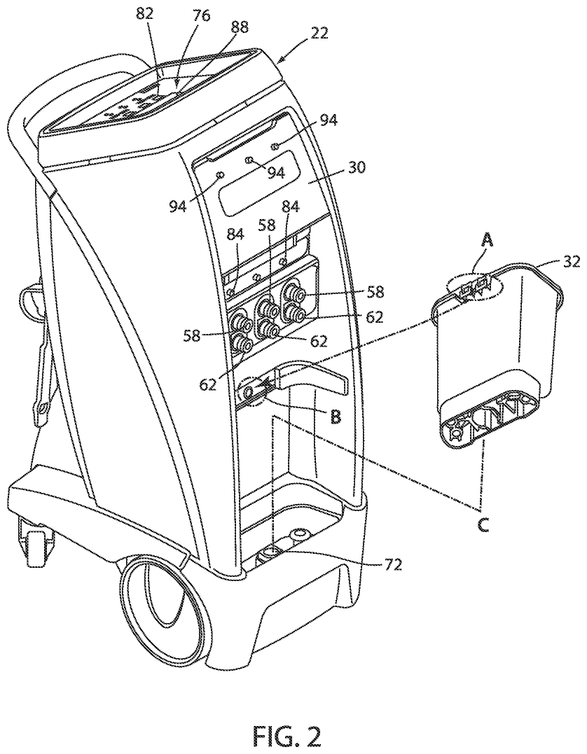

As shown more clearly in FIG. 2, thermal control unit 22 includes a main body 30 to which a removable reservoir 32 may be coupled and uncoupled. Removable reservoir 32 is configured to hold the fluid that is to be circulated through thermal control unit 22 and the one or more thermal pads 24. By being removable from thermal control unit 22, reservoir 32 can be easily carried to a sink or faucet for filling and/or dumping of the water or other fluid. This allows users of thermal control system 20 to more easily fill thermal control unit 22 prior to its use, as well as to drain thermal control unit 22 after use.

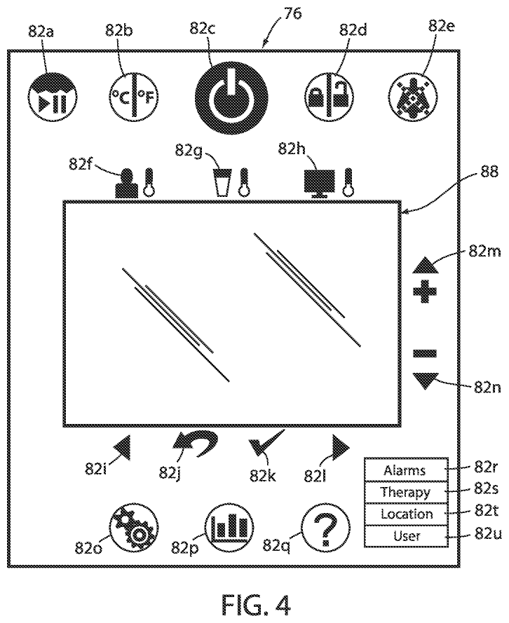

As can also be seen in FIG. 2, thermal control unit 22 includes a plurality of outlet ports 58 (three in the particular example of FIG. 2), a plurality of inlet ports 62 (three in this particular example). Outlet ports 58 are adapted to fluidly couple to supply hoses 26a and inlet ports are adapted to fluidly couple to return hoses 26b. Thermal control unit 22 also includes a plurality of patient temperature probe ports 84, a plurality of auxiliary ports 94, and a control panel 76 having a plurality of dedicated controls 82 and a display 88 (see also FIG. 4). The patient temperature probe ports 84, auxiliary ports 94, and control panel 76 are described in more detail below.

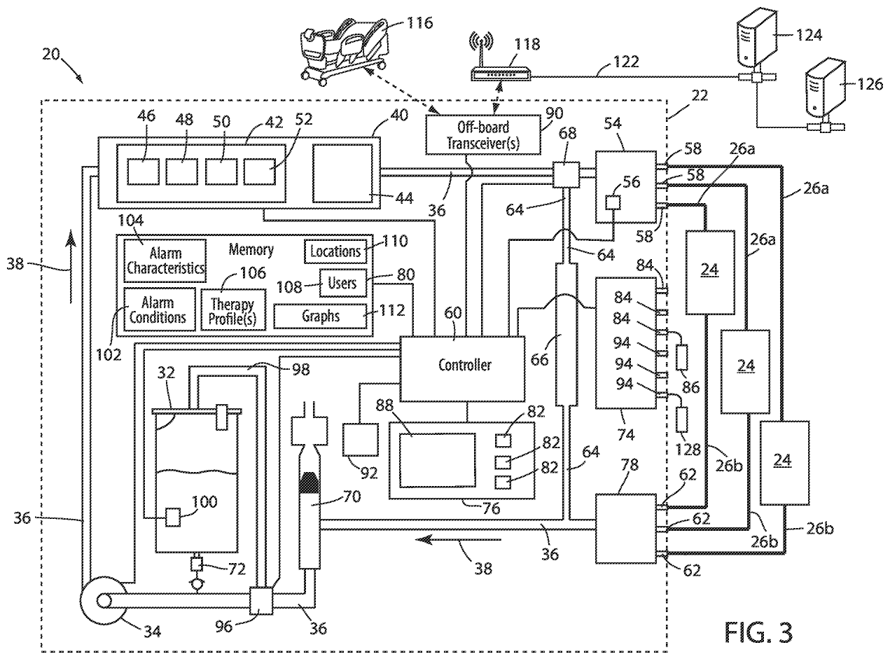

As shown in FIG. 3, thermal control unit 22 includes a pump 34 for circulating fluid through a circulation channel 36. Pump 34, when activated, circulates the fluid through circulation channel 36 in the direction of arrows 38 (clockwise in FIG. 3). Starting at pump 34 the circulating fluid first passes through a heat exchanger 40 that adjusts, as necessary, the temperature of the circulating fluid. Heat exchanger 40 may take on a variety of different forms. In some embodiments, heat exchanger 40 is a thermoelectric heater and cooler. In the embodiment shown in FIG. 3, heat exchanger 40 includes a chiller 42 and a heater 44. Further, in the embodiment shown in FIG. 3, chiller 42 is a conventional vapor-compression refrigeration unit having a compressor 46, a condenser 48, an evaporator 50, an expansion valve (not shown), and a fan 52 for removing heat from the compressor 46. Heater 44 is a conventional electrical resistance-based heater. Other types of chillers and/or heaters may be used.

After passing through heat exchanger 40, the circulating fluid is delivered to an outlet manifold 54 having an outlet temperature sensor 56 and a plurality of outlet ports 58. Temperature sensor 56 is adapted to detect a temperature of the fluid inside of outlet manifold 54 and report it to a controller 60. Outlet ports 58 are coupled to supply hoses 26a. Supply hoses 26a are coupled, in turn, to thermal pads 24 and deliver temperature-controlled fluid to the thermal pads 24. The temperature-controlled fluid, after passing through the thermal pads 24, is returned to thermal control unit 22 via return hoses 26b. Return hoses 26b couple to a plurality of inlet ports 62. Inlet ports 62 are fluidly coupled to an inlet manifold 78 inside of thermal control unit 22.

Thermal control unit 22 also includes a bypass line 64 fluidly coupled to outlet manifold 54 and inlet manifold 78 (FIG. 3). Bypass line 64 allows fluid to circulate through circulation channel 36 even in the absence of any thermal pads 24 or hoses 26a being coupled to any of outlet ports 58. In the illustrated embodiment, bypass line 64 includes a filter 66 that is adapted to filter the circulating fluid. If included, filter 66 may be a particle filter adapted to filter out particles within the circulating fluid that exceed a size threshold, or filter 66 may be a biological filter adapted to purify or sanitize the circulating fluid, or it may be a combination of both. In some embodiments, filter 66 is constructed and/or positioned within thermal control unit 22 in any of the manners disclosed in commonly assigned U.S. patent application Ser. No. 62/404,676 filed Oct. 11, 2016, by inventors Marko Kostic et al. and entitled THERMAL CONTROL SYSTEM, the complete disclosure of which is incorporated herein by reference.

The flow of fluid through bypass line 64 is controllable by way of a bypass valve 68 positioned at the intersection of bypass line 64 and outlet manifold 54 (FIG. 3). When open, bypass valve 68 allows fluid to flow through circulation channel 36 to outlet manifold 54, and from outlet manifold 54 to the connected thermal pads 24. When closed, bypass valve 68 stops fluid from flowing to outlet manifold 54 (and thermal pads 24) and instead diverts the fluid flow along bypass line 64. In some embodiments, bypass valve 68 may be controllable such that selective portions of the fluid are directed to outlet manifold 54 and along bypass line 64. In some embodiments, bypass valve 68 is controlled in any of the manners discussed in commonly assigned U.S. patent application Ser. No. 62/610,319, filed Dec. 26, 2017, by inventors Gregory Taylor et al. and entitled THERMAL SYSTEM WITH OVERSHOOT REDUCTION, the complete disclosure of which is incorporated herein by reference. In other embodiments, bypass valve 68 may be a pressure operated valve that allows fluid to flow along bypass line 64 if the fluid pressure in circulation channel 36 exceeds the cracking pressure of the bypass valve 68. Still further, in some embodiments, bypass valve 68 may be omitted and fluid may be allowed to flow through both bypass line 64 and into outlet manifold 54.

The incoming fluid flowing into inlet manifold 78 from inlet ports 62 and/or bypass line 64 travels back toward pump 34 and into an air remover 70. Air remover 70 includes any structure in which the flow of fluid slows down sufficiently to allow air bubbles contained within the circulating fluid to float upwardly and escape to the ambient surroundings. In some embodiments, air remover 70 is constructed in accordance with any of the configurations disclosed in commonly assigned U.S. patent application Ser. No. 15/646,847 filed Jul. 11, 2017, by inventor Gregory S. Taylor and entitled THERMAL CONTROL SYSTEM, the complete disclosure of which is hereby incorporated herein by reference. After passing through air remover 70, the circulating fluid flows past a valve 72 positioned beneath fluid reservoir 32. Fluid reservoir 32 supplies fluid to thermal control unit 22 and circulation channel 36 via valve 72, which may be a conventional check valve, or other type of valve, that automatically opens when reservoir 32 is coupled to thermal control unit 22 and that automatically closes when reservoir 32 is decoupled from thermal control unit 22 (see FIG. 2). After passing by valve 72, the circulating fluid travels to pump 34 and the fluid circuit is repeated.

Controller 60 of thermal control unit 22 is contained within main body 30 of thermal control unit 22 and is in electrical communication with pump 34, heat exchanger 40, outlet temperature sensor 56, bypass valve 68, a sensor module 74, control panel 76, a memory 80, one or more transceivers 90, and, in some embodiments, one or more other sensors, such as, but not limited to, a location sensor 92. Controller 60 includes any and all electrical circuitry and components necessary to carry out the functions and algorithms described herein, as would be known to one of ordinary skill in the art. Generally speaking, controller 60 may include one or more microcontrollers, microprocessors, and/or other programmable electronics that are programmed to carry out the functions described herein. It will be understood that controller 60 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in thermal control unit 22, or they may reside in a common location within thermal control unit 22. When physically distributed, the components may communicate using any suitable serial or parallel communication protocol, such as, but not limited to, CAN, LIN, Firewire, I-squared-C, RS-232, RS-465, universal serial bus (USB), etc.

Control panel 76 allows a user to operate thermal control unit 22. Control panel 76 communicates with controller 60 and includes a display 88 and a plurality of dedicated controls 82a, 82b, 82c, etc. Display 88 may be implemented as a touch screen, or, in other embodiments, as a non-touch-sensitive display. Dedicated controls 82 may be implemented as buttons, switches, dials, or other dedicated structures. In any of the embodiments, one or more of the functions carried out by a dedicated control 82 may be replaced or supplemented with a touch screen control that is activated when touched by a user. Alternatively, in any of the embodiments, one or more of the controls that are carried out via a touch screen can be replaced or supplemented with a dedicated control 82 that carries out the same function when activated by a user.

Through either dedicated controls 82 and/or a touch screen display (e.g. display 88), control panel 76 enables a user to turn thermal control unit 22 on and off, select a mode of operation, select a target temperature for the fluid delivered to thermal pads 24, select a patient target temperature, customize a variety of treatment, display, alarm, and other functions, and control still other aspects of thermal control unit 22, as is discussed in greater detail below. In some embodiments, control panel 76 may include a pause/event control, a medication control, and/or an automatic temperature adjustment control that operate in accordance with the pause event control 66b, medication control 66c, and automatic temperature adjustment control 66d disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. Such controls may be activated as touch screen controls or dedicated controls 82.

In those embodiments where control panel 76 allows a user to select from different modes for controlling the patient's temperature, the different modes include, but are not limited to, a manual mode and an automatic mode, both of which may be used for cooling and heating the patient. In the manual mode, a user selects a target temperature for the fluid that circulates within thermal control unit 22 and that is delivered to thermal pads 24. Thermal control unit 22 then makes adjustments to heat exchanger 40 in order to ensure that the temperature of the fluid exiting supply hoses 26a is at the user-selected temperature.

When the user selects the automatic mode, the user selects a target patient temperature, rather than a target fluid temperature. After selecting the target patient temperature, controller 60 makes automatic adjustments to the temperature of the fluid in order to bring the patient's temperature to the desired patient target temperature. In this mode, the temperature of the circulating fluid may vary as necessary in order to bring about the target patient temperature.

In order to carry out the automatic mode, thermal control unit 22 utilizes a sensor module 74 that includes one or more patient temperature sensor ports 84 (FIGS. 2 & 3) that are adapted to receive one or more conventional patient temperature sensors or probes 86. The patient temperature sensors 86 may be any suitable patient temperature sensor that is able to sense the temperature of the patient at the location of the sensor. In one embodiment, the patient temperature sensors are conventional Y.S.I. 400 probes marketed by YSI Incorporated of Yellow Springs, Ohio, or probes that are YSI 400 compliant or otherwise marketed as 400 series probes. In other embodiments, different types of sensors may be used with thermal control unit 22. Regardless of the specific type of patient temperature sensor used in thermal control system 20, each temperature sensor 86 is connected to a patient temperature sensor port 84 positioned on thermal control unit 22. Patient temperature sensor ports 84 are in electrical communication with controller 60 and provide current temperature readings of the patient's temperature.

Controller 60, in some embodiments, controls the temperature of the circulating fluid using closed-loop feedback from temperature sensor 56 (and, when operating in the automatic mode, also from patient temperature sensor(s) 86). That is, controller 60 determines (or receives) a target temperature of the fluid, compares it to the measured temperature from sensor 56, and issues a command to heat exchanger 40 that seeks to decrease the difference between the desired fluid temperature and the measured fluid temperature. In some embodiments, the difference between the fluid target temperature and the measured fluid temperature is used as an error value that is input into a conventional Proportional, Integral, Derivative (PID) control loop. That is, controller 60 multiplies the fluid temperature error by a proportional constant, determines the derivative of the fluid temperature error over time and multiplies it by a derivative constant, and determines the integral of the fluid temperature error overtime and multiplies it by an integral constant. The results of each product are summed together and converted to a heating/cooling command that is fed to heat exchanger 40 and tells heat exchanger 40 whether to heat and/or cool the circulating fluid and how much heating/cooling power to use.

When thermal control unit 22 is operating in the automatic mode, controller 60 may use a second closed-loop control loop that determines the difference between a patient target temperature and a measured patient temperature. The patient target temperature is input by a user of thermal control unit 22 using control panel 76. The measured patient temperature comes from a patient temperature sensor 86 coupled to one of patient temperature sensor ports 84 (FIG. 3). Controller 60 determines the difference between the patient target temperature and the measured patient temperature and, in some embodiments, uses the resulting patient temperature error value as an input into a conventional PID control loop. As part of the PID loop, controller 60 multiplies the patient temperature error by a proportional constant, multiplies a derivative of the patient temperature error over time by a derivative constant, and multiplies an integral of the patient temperature error over time by an integral constant. The three products are summed together and converted to a target fluid temperature value. The target fluid temperature value is then fed to the first control loop discussed above, which uses it to compute a fluid temperature error.

It will be understood by those skilled in the art that other types of control loops may be used. For example, controller 60 may utilize one or more PI loops, PD loops, and/or other types of control equations. In some embodiments, the coefficients used with the control loops may be varied by controller 60 depending upon the patient's temperature reaction to the thermal therapy, among other factors. One example of such dynamic control loop coefficients is disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference.

Regardless of the specific control loop utilized, controller 60 implements the loop(s) multiple times a second in at least one embodiment, although it will be understood that this rate may be varied widely. After controller 60 has output a heat/cool command to heat exchanger 40, controller 60 takes another patient temperature reading (from sensor 86) and/or another fluid temperature reading (from sensor 56) and re-performs the loop(s). The specific loop(s) used, as noted previously, depends upon whether thermal control unit 22 is operating in the manual mode or automatic mode.

It will also be understood by those skilled in the art that the output of any control loop used by thermal control unit 22 may be limited such that the temperature of the fluid delivered to thermal pads 24 never strays outside of a predefined maximum and a predefined minimum. Examples of such a predefined maximum temperature and predefined minimum temperature are disclosed and discussed in greater detail in commonly assigned U.S. patent application Ser. No. 16/222,004 filed Dec. 17, 2018, by inventors Gregory S. Taylor et al. and entitled THERMAL SYSTEM WITH GRAPHICAL USER INTERFACE, the complete disclosure of which is incorporated herein by reference. The predefined minimum temperature is designed as a safety temperature and may be set to about four degrees Celsius, although other temperatures may be selected. The predefined maximum temperature is also implemented as a safety measure and may be set to about forty degrees Celsius, although other values may be selected.

In some embodiments of thermal control unit 22, such as the embodiment shown in FIG. 3, thermal control unit 22 also includes a reservoir valve 96 that is adapted to selectively move fluid reservoir 32 into and out of line with circulation channel 36. Reservoir valve 96 is positioned in circulation channel 36 between air remover 70 and valve 72, although it will be understood that reservoir valve 96 may be moved to different locations within circulation channel 36. Reservoir valve 96 is coupled to circulation channel 36 as well as a reservoir channel 98. When reservoir valve 96 is open, fluid from air remover 70 flows along circulation channel 36 to pump 34 without passing through reservoir 32 and without any fluid flowing along reservoir channel 98. When reservoir valve 96 is closed, fluid coming from air remover 70 flows along reservoir channel 98, which feeds the fluid into reservoir 32. Fluid inside of reservoir 32 then flows back into circulation channel 36 via valve 72. Once back in circulation channel 36, the fluid flows to pump 34 and is pumped to the rest of circulation channel 36 and thermal pads 24 and/or bypass line 64. In some embodiments, reservoir valve 96 is either fully open or fully closed, while in other embodiments, reservoir valve 96 may be partially open or partially closed. In either case, reservoir valve 96 is under the control of controller 60.

In those embodiments of thermal control unit 22 that include a reservoir valve, thermal control unit 22 may also include a reservoir temperature sensor 100. Reservoir temperature sensor 100 reports its temperature readings to controller 60. When reservoir valve 96 is open, the fluid inside of reservoir 32 stays inside of reservoir 32 (after the initial drainage of the amount of fluid needed to fill circulation channel 36 and thermal pads 24). This residual fluid is substantially not affected by the temperature changes made to the fluid within circulation channel 36 as long as reservoir valve 96 remains open. This is because the residual fluid that remains inside of reservoir 32 after circulation channel 36 and thermal pads 24 have been filled does not pass through heat exchanger 40 and remains substantially thermally isolated from the circulating fluid. Two results flow from this: first, heat exchanger 40 does not need to expend energy on changing the temperature of the residual fluid in reservoir 32, and second, the temperature of the circulating fluid in circulation channel 36 will deviate from the temperature of the residual fluid as the circulating fluid circulates through heat exchanger 40.

In some embodiments, controller 60 utilizes a temperature control algorithm to control reservoir valve 96 that, in some embodiments, is the same as the temperature control algorithm 160 disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. In other embodiments, controller 60 utilizes a different control algorithm. In still other embodiments, thermal control unit 22 is modified to omit reservoir valve 96, reservoir channel 98, and reservoir temperature sensor 100. Thermal control unit 22 may also be modified such that reservoir 32 is always in the path of circulation channel 36. Still other modifications are possible.

It will be understood that the particular order of the components along circulation channel 36 of thermal control unit 22 may be varied from what is shown in FIG. 3. For example, although FIG. 3 depicts pump 34 as being upstream of heat exchanger 40 and air separator 70 as being upstream of pump 34, this order may be changed. Air separator 70, pump 34, heat exchanger 40 and reservoir 32 may be positioned at any suitable location along circulation channel 36. Indeed, in some embodiments, reservoir 32 is moved so as to be in line with and part of circulation channel 36, rather than external to circulation channel 36 as shown in FIG. 3, thereby forcing the circulating fluid to flow through reservoir 32 rather than around reservoir 32. It will also be understood that thermal control unit 22 does not need to include all of the components shown in FIG. 3, and that many embodiments of thermal control unit 22 may be implemented in accordance with the present disclosure that omit one or more of these illustrated components. Further details regarding the construction and operation of one embodiment of thermal control unit 22 that are not described herein may be found in commonly assigned U.S. patent application Ser. No. 14/282,383 filed May 20, 2014, by inventors Christopher Hopper et al. and entitled THERMAL CONTROL SYSTEM, the complete disclosure of which is incorporated herein by reference.

In some embodiments, thermal pads 24 are constructed in accordance with any of the thermal pads disclosed in any of the following commonly assigned U.S. patent applications: Ser. No. 15/675,061 filed Aug. 11, 2017, by inventors James Galer et al. and entitled THERMAL THERAPY DEVICES; Ser. No. 62/778,034 filed Dec. 11, 2018, by inventors Andrew M. Bentz et al. and entitled THERMAL SYSTEM WITH THERMAL PAD FILTERS; and Ser. No. 15/675,066 filed Aug. 11, 2017, by inventor James K. Galer and entitled THERMAL SYSTEM, the complete disclosures of all of which are incorporated herein by reference. Still other types of thermal pads 24 may be used with thermal control system 20, and thermal control unit 22 may be modified from its construction described herein in order to accommodate the particular thermal therapy pad(s) it is used with.

Memory 80 (FIG. 3) may be any type of conventional non-volatile memory, such as, but not limited to flash memory, one or more hard drives, one or more EEPROMs, etc. Memory 80 may also be implemented to include more than one of these types of memories in combination. In the embodiment shown in FIG. 3, memory 80 of thermal control unit 22 includes a plurality of items stored therein, such as one or more sets of each of the following: alarm conditions 102, alarm characteristics 172, therapy profiles 106, user data 108, location data 110, and graphing data 112. These items are able to be entered into memory 80 locally via control panel 76 and/or are written into memory 80 by controller 60. Additionally, in some embodiments, any of these items in memory 80 may be transferred (wired or wirelessly) to thermal control unit 22 from another device, such as, but not limited to, a server, another thermal control unit, a flash drive, a patient support apparatus 116 on which patient 28 is lying (see FIG. 1), one or more other devices, and/or a combination of any of the aforementioned devices. Such data transfers may take place via transceiver 90. Memory 80 may also include additional information beyond that shown in FIG. 3, such as, but not limited to, one or more algorithms for carrying out its functions, data recorded during the operation of thermal control unit 22, and/or other data. Memory 80 may also, in some embodiments, omit any one or more of the specific data items shown in FIG. 3.

Off-board transceiver 90 is adapted to communicate with one or more off-board devices, such as, but not limited to, a wireless access point of local area network, a network cable of a local area network, and/or other devices. In the embodiment shown in FIG. 3, transceiver 90 is a Wi-Fi radio communication module configured to wirelessly communicate with one or more wireless access points 118 of a local area network 122. In such embodiments, transceiver 90 may operate in accordance with any of the various IEEE 802.11 standards (e.g. 802.11b, 802.11n, 802.11g, 802.11ac, 802.11ah, etc.). In other embodiments, transceiver 90 may include, either additionally or in lieu of the Wi-Fi radio and communication module, a wired port for connecting a network wire to thermal control unit 22. In some such embodiments, the wired port accepts a category 5e cable (Cat-5e), a category 6 or 6a (Cat-6 or Cat-6a), a category 7 (Cat-7) cable, or some similar network cable, and transceiver 90 is an Ethernet transceiver. In still other embodiments, transceiver 90 may be constructed to include the functionality of the communication modules 56 disclosed in commonly assigned U.S. patent application Ser. No. 15/831,466 filed Dec. 5, 2017, by inventor Michael Hayes et al. and entitled NETWORK COMMUNICATION FOR PATIENT SUPPORT APPARATUSES, the complete disclosure of which is incorporated herein by reference.

Regardless of the specific structure included with transceiver 90, controller 60 is able to communicate with the local area network 122 (FIG. 3) of a healthcare facility in which the thermal control unit 22 is positioned. When transceiver 90 is a wireless transceiver, it communicates with local area network 122 via one or more wireless access points 118. When transceiver 90 is a wired transceiver, it communicates directly via a cable coupled between thermal control unit 22 and a network outlet positioned within the room of the healthcare facility in which thermal control unit 22 is positioned.

Local area network 122 typically includes a plurality of servers, the contents of which will vary from healthcare facility to healthcare facility. In general, however, most healthcare facilities will include, among other servers, an electronic medical records (EMR) server 124, which may be a conventional server. In addition to EMR server 124, local area network 122 includes a remote computing device or remote server 126 (the two terms are used interchangeably herein) that is in communication with one or more thermal control units 22 positioned within the healthcare facility. Remote computing device 126 may also be communicatively coupled (via the Internet or other means) to one or more other servers that are positioned outside of the healthcare facility.

In addition to the aforementioned servers 124 and 126, one or more additional servers may also be included, such as, but not limited to, an Internet server and/or an Internet gateway that couples network 122 to the Internet, thereby enabling remote computing device 126, thermal control units 22, and/or other applications on network 122 to communicate with computers outside of the healthcare facility, such as, but not limited to, a geographically remote server operated under the control of the manufacturer of thermal control units 22. Another type of server that may be included with computer network 122 is a location server (not shown) that is adapted to monitor and record the current locations of thermal control units 22, patients, and/or caregivers within the healthcare facility. Such a location server communicates with the thermal control units 22 via access points 118 and transceivers 90.

Network 122 may also include a conventional Admission, Discharge, and Tracking (ADT) server that allows thermal control units 22 to retrieve information identifying the patient undergoing thermal therapy. Still further, healthcare network 122 may further include one or more conventional work flow servers and/or charting servers that assign, monitor, and/or schedule patient-related tasks to particular caregivers, and/or one or more conventional communication servers that forward communications to particular individuals within the healthcare facility, such as via one or more portable devices (smart phones, pagers, beepers, laptops, etc.). The forwarded communications may include data and/or alerts that originate from thermal control units 22 and/or elsewhere.

In some embodiments, local area network 122 may include any one or more of the servers described and disclosed in commonly assigned PCT patent application serial number PCT/US2020/039587 filed Jun. 25, 2020, by inventors Thomas Durlach et al. and entitled CAREGIVER ASSISTANCE SYSTEM, the complete disclosure of which is incorporated herein by reference. Further, in in such embodiments, thermal control units 22 may be configured to communicate with the servers on network 122 in any of the manners disclosed in the '587 PCT application, and/or to retrieve and/or share any of the information disclosed in the '587 PCT application.

Although not shown in FIG. 3, thermal control unit 22 includes a clock/calendar that communicates with controller 60. The clock/calendar not only measures the passage of time, but it also keeps track of the calendar day (and year). As will be discussed in greater detail below, controller 60 may use the outputs from clock/calendar day when it gathers data for improving one or more algorithms followed by controller 60, and/or when it automatically implements one or more user-preferred settings. The clock/calendar may be any conventional timing device that is able to keep track of the passage of time, including the calendar day and year.

In the embodiment shown in FIG. 3, thermal control unit 22 further includes a location sensor 92. Location sensor 92 automatically detects the location of thermal control unit 22 within a healthcare facility. Location sensor 92 may take on a variety of different forms. For example, in one embodiment, thermal control unit 22 includes a WiFi transceiver (which may be the same as transceiver 90 or may be an additional/separate transceiver) that communicates with the healthcare facility's local area network via the network's wireless access points 118, and controller 60 determines its location relative to the known locations of these access points based upon the detected signal strengths from these access points. In another example, location sensor 92 and controller 60 may determine their location using any of the same methods and/or sensors for determining patient support apparatus location that are disclosed in commonly assigned U.S. Pat. No. 9,838,836 issued Dec. 5, 2017, to inventors Michael J. Hayes et al. and entitled PATIENT SUPPORT APPARATUS COMMUNICATION SYSTEMS, the complete disclosure of which is incorporated herein by reference. Still other automatic location detection methods may be used, including, but not limited to, the use of cellular network trilateration and/or Global Positioning System (GPS) sensors.

In addition to the patient temperature sensor(s) 86, the water temperature sensor 56, the reservoir temperature sensor 100 (if included), and the location sensor 92 (if included), thermal control unit 22 may include still more sensors that are positioned within main body 30, and/or that are positioned outside of main body 30 and in communication with main controller 60. Such off-board sensors (e.g. outside of main body 30) may communicate with main controller 60 via one or more of the auxiliary sensor ports 94 and/or via one or more of the transceivers 90. Each auxiliary sensor port 94 is adapted to receive outputs from an off-board auxiliary sensor 128. The auxiliary sensors 128, as well as any additional sensors onboard thermal control unit 22, provide additional data to controller 60 regarding the patient during a thermal therapy session. Controller 60 is configured to utilize the additional data either for use in one or more algorithms that are currently being used by thermal control unit 22 to control the patient's temperature, or for potential future use in one or more improved algorithms that are determined, after analysis, to provide improved results for the thermal therapy sessions carried out using thermal control unit 22. As will be discussed in greater detail below, the additional data, when not currently used for controlling thermal control unit 22, may be analyzed by remote computing device 126 in order to determine if the additional data can improve the performance of thermal control unit 22, and/or if it can predict the occurrence of one more undesired events, such as, for example, patient shivering or overshooting the target temperature of the patient. These purposes will be discussed in greater detail below.

Auxiliary ports 94 (FIGS. 2 & 3) may take on a variety of different forms. In one embodiment, all of the ports 94 (if there are more than one) are of the same type. In another embodiment, thermal control unit 22 includes multiple types of ports. In any of these embodiments, the ports 94 may include, but are not limited to, a Universal Serial Bus (USB) port, an Ethernet port (e.g. an 8P8C modular connector port, or the like), a parallel port, a different (from USB) type of serial port, etc. Ports 94 may also or alternatively be implemented wirelessly, such as via a WiFi transceiver, a Bluetooth transceiver, a ZigBee transceiver, etc. In these latter embodiments, one or more of transceivers 90 may be incorporated into sensor module 74 and in communication with one or more of the ports 94.

Thermal control unit 22 is configured to accept a number of different types of auxiliary sensors 128 via input ports 94. Such sensors include, but are not limited to, the following: an end tidal carbon dioxide (ETCO₂) sensor that detects ETCO₂ levels of the patient; a respiration rate sensor that senses the respiration rate of the patient; a blood pressure sensor that detects the blood pressure of the patient; a heart rate sensor that detects the heart rate of the patient; a scale sensor that detects the patient's weight and/or movement; an electrolyte sensor that detects levels of one or more electrolytes (e.g. potassium) in the patient's blood; a pulse wave velocity sensor that detects the patient's pulse wave velocity; an oxygen saturation level (SpO2) sensor that detect oxygen saturation levels of the patient; a bioimpedance sensor that detects a bioimpedance of the patient, such as, but not limited to, the bioimpedance at one or more locations on the patient's body in contact with a thermal pad 24; an electrocardiograph sensor that detects an electrocardiogram of the patient; a temperature change sensor that detects a rate of temperature change of the patient; one or more sensors that are integrated into one or more of the thermal pads 24 and that detect characteristics of the thermal pads 24 and/or of the patient (e.g. temperature sensors built into the thermal pads 24); one or more temperature sensors that detect one or more peripheral temperatures of the patient (as opposed to the core temperature sensed by sensor 86); an ultrasonic sensor adapted to detect attenuation levels of ultrasonic waves traveling through at least a portion of the patient's body; a near infrared sensor adapted to detect attenuation levels of near infrared waves traveling through at least a portion of the patient's body; a perfusion sensor adapted to detect a patient's blood perfusion levels; a vibration sensor (e.g. accelerometer) adapted to detect vibrations of the patient, such as due to shivering; a thermal image sensor adapted to capture thermal images of the patient; an electromyograph adapted to detect electrical activity in the patient's muscles; one or more air quality sensors (e.g. air pressure, humidity, air temperature, air volume, etc.) that measure characteristics of the air breathed by the patient and/or the ambient air; and/or still other sensors.

When thermal control unit 22 is utilized with a respiration rate sensor and/or a heart rate sensor coupled to one or more auxiliary ports 94, these sensors may be directly attached to the patient and/or they may be adapted to passively monitor these parameters without direct attachment to the patient. In some embodiments, passive heart rate sensors and/or respiration rate sensors may be built directly into patient support apparatus 116 that communicate their outputs to thermal control unit 22. One example of such sensor are disclosed in commonly assigned U.S. Pat. No. 7,699,784 filed Jul. 5, 2007, by inventors David Wan Fong et al. and entitled SYSTEM FOR DETECTING AND MONITORING VITAL SIGNS, the complete disclosure of which is incorporated herein by reference. Still other types of both passive and non-passive vital sign sensors may be used.

When thermal control unit 22 is utilized with any one or more of an end tidal carbon dioxide (ETCO₂) sensor, a blood pressure sensor, an oxygen saturation level sensor, a respiration rate sensor, a heart rate sensor, an electrolyte sensor, a pulse wave velocity sensor, a bioimpedance sensor, an electrocardiograph sensor, or a rate of temperature change sensor coupled to one or more auxiliary ports 94, such sensors may be of the same type, and/or utilized in the same or similar manners, as those disclosed in more detail in commonly assigned U.S. patent application Ser. No. 16/912,244 filed Jun. 25, 2020, by inventors Gregory S. Taylor et al. and entitled THERMAL SYSTEM WITH USER INTERFACE CUSTOMIZATION, the complete disclosure of which is incorporated herein by reference. Still other types of these sensors may be used.

When thermal control unit 22 is utilized with any one or more sensors that are integrated into one or more of the thermal pads 24 and that are coupled to one or more auxiliary ports 94, such sensors may be of the same type, and/or utilized in the same or similar manners, as those disclosed in more detail in commonly assigned U.S. patent application Ser. No. 15/675,066 filed Aug. 11, 2017, by inventor James Galer and entitled THERMAL SYSTEM, the complete disclosure of which is incorporated herein by reference. Still other types of these sensors may be used.

When thermal control unit 22 is utilized with any one or more of an ultrasonic sensor, an infrared sensor, a perfusion sensor, and/or a peripheral patient temperature sensor coupled to one or more auxiliary ports 94, such sensors may be of the same type, and/or utilized in the same or similar manners, as those disclosed in more detail in commonly assigned PCT patent application PCT/US2018/066114 filed Dec. 18, 2018, by Applicant Stryker Corporation and entitled THERMAL SYSTEM WITH PATIENT SENSOR(S), the complete disclosure of which is incorporated herein by reference. Still other types of these sensors may be used.

When thermal control unit 22 is utilized with any one or more of a vibration sensor, a thermal image sensor, and/or an electromyograph coupled to one or more auxiliary ports 94, such sensors may be of the same type, and/or utilized in the same or similar manners, as those disclosed in more detail in commonly assigned U.S. patent application Ser. No. 15/820,558 filed Nov. 22, 2017, by inventors Gregory S. Taylor et al. and entitled THERMAL SYSTEM, the complete disclosure of which is incorporated herein by reference. Still other types of these sensors may be used.

When thermal control unit 22 is utilized with any one or more air quality sensors (air pressure, humidity, air temperature, air volume, etc.) coupled to one or more auxiliary ports 94, such sensors may be of the same type, and/or utilized in the same or similar manners, as those disclosed in more detail in commonly assigned PCT patent application PCT/US2018/064685 filed Dec. 10, 2018, by Applicant Stryker Corporation and entitled THERMAL CONTROL SYSTEM, the complete disclosure of which is incorporated herein by reference. Still other types of these sensors may be used.

When thermal control unit 22 is utilized with a scale sensor coupled to one or more auxiliary ports 94, the scale sensor may be built into patient support apparatus 116 and/or separate from patient support apparatus 116. In some embodiments where the scale sensor is built into patient support apparatus 116, the scale sensor may include any of the load cells and/or other movement sensors disclosed in commonly assigned U.S. patent application Ser. No. 14/873,734 filed Oct. 2, 2015, by inventors Marko Kostic et al. and entitled PERSON SUPPORT APPARATUSES WITH MOTION MONITORING, and/or in commonly assigned U.S. patent application Ser. No. 15/346,779 filed Nov. 9, 2016, by inventors Marko Kostic et al. and entitled PERSON SUPPORT APPARATUSES WITH ACCELERATION DETECTION, the complete disclosures of both of which are incorporated herein by reference.

It will be understood that the sensors incorporated into thermal control unit 22 may be augmented and/or otherwise modified from what is shown in FIG. 3. For example, thermal control unit 22 may include one or more of the following sensors positioned inside main body 30: one or more input fluid temperature sensors that measure the temperature of the fluid returning to inlet manifold 78 (e.g. a single inlet temperature sensor or multiple inlet temperature sensors that measure the fluid temperature for each individual inlet port 62); one or more water quality sensors that measure the cleanliness and/or other characteristics of the fluid (e.g. water) that is circulating in circulation channel 36 and being delivered to the thermal pads 24; one or more flow rate sensors that measure the flow rate of fluid through circulation channel 36 (and/or that measure individual flow rates through each of the outlet ports 58 and/or through each of the inlet ports 62); one or more valve sensors that detect the position of one or more valves within thermal control unit 22; and/or still other types of sensors.

It will also be understood that the terms “sensor” and “sensors” as used herein may also refer to devices that store data and that communicate the stored data to thermal control unit 22, either through one or more ports 94 and/or through one or more of the transceivers 90. Such storage devices include, but are not limited to, EMR server 124, remote computing device 126, and any other servers that may be in communication with local area network 122. In addition, such other storage devices include patient support apparatus 116, any portable electronic devices carried by a caregiver (e.g. a smart phone, tablet computer, laptop computer, etc.), and/or other types of devices that are capable of storing data relevant to patient 28 and/or his or her thermal therapy session. The data communicated to thermal control unit 22 from these storage devices may include any one or more of the following: the patient's age, weight, height, BMI, BSA, and/or other patient information; medication information indicating what medications patient 28 is on or has received prior to, or during, the thermal therapy session; location information that indicates the current location of thermal control unit 22; caregiver identification information that identifies which caregiver is currently using thermal control unit 22; treatment information identifying the diagnosis of patient 28 and/or the intended use for the thermal therapy session (e.g. for neurotrauma, cardiac arrest, etc.); and/or still other types of information.

Still further, it will be understood that, in addition to the aforementioned sensors, thermal control unit 22 may include within main body 30, and/or be in communication with, one or more meta-sensors that detect characteristics of any one or more of the aforementioned sensors. Examples of suites of meta-sensors that are used to detect the condition of one or more other sensors onboard a patient support apparatus are disclosed in commonly assigned U.S. patent application Ser. No. 16/367,872 filed Mar. 28, 2019 by inventors Marko Kostic et al. and entitled PATIENT SUPPORT APPARATUSES WITH MULTI-SENSOR FUSION, the complete disclosure of which is incorporated herein by reference. Any of the meta-sensors disclosed in this reference may be utilized onboard thermal control unit 22 and/or with any of the devices in communication with thermal control unit 22. Still other types of meta-sensors may be used.

FIG. 4 shows one manner in which the layout of control panel 76 can be implemented, including an illustrative arrangement of dedicated controls 82 that are positioned around display 88. Controls 82 include a therapy pause control 82a that, when pressed, pauses the therapy being performed by thermal control unit 22. To resume therapy, a user presses and holds down on the therapy pause control 82. A selection control 82b allows a user to switch between displaying the temperatures in Fahrenheit and Celsius by pressing on control 82b, which acts as a toggle switch between the two different units of measurement. A power control 82c will turn on and off thermal control unit 22 when pressed. When a user first presses a lock control 82d, the screen will be locked and pressing on any areas of the screen will not change any settings, or otherwise cause thermal control unit 22 to react to the pressing. In order to unlock the touchscreen, a user presses down and holds the lock control 82d for at least two seconds. An audio pause control 82e, when pressed, silences any audible alarms for a predetermined period of time, such as ten minutes. Any alarms will still result in a visual display of the alarm on display 88, but will not result in any audible indications while the audio pause is in effect.

Control panel 76 further includes three therapy mode controls 82f, 82g, and 82h. Pushing down on control 82f will cause thermal control unit 22 to act in the automatic mode (described previously). Pushing down on control 82g will cause thermal control unit 22 to act in the manual mode (also described previously). Pushing down on control 82g will cause thermal control unit 22 to act in a monitor mode (not described previously). In the monitor mode, thermal control unit 22 does not circulate fluid or regulate the fluid's temperature, but instead merely monitors the temperature(s) input into thermal control unit 22 via the patient temperature probe ports 84 and issues any alarms if the temperatures change beyond any user-defined thresholds.

A back control 82i causes controller 60 to change what is displayed on display 88 to that which was displayed thereon immediately prior to the pressing of the back control 82i. An edit control 82j enables the user to edit current settings when pressed, or exit or cancel, depending upon the context of the information displayed on LCD display 88. A confirm control 82k, when pressed, allows a user to confirm a selection made by the user of information displayed on display 88. A forward control 82l shifts, when pressed, what is displayed on display 88 to the next sequential screen.

Controls 82m and 82n enable a user to increase or decrease a patient or fluid temperature, depending upon the context of what is displayed on display 88. Control 82o is a settings icon that, when pressed, displays a summary of the current settings of thermal control unit 22, and may also display controls for changing one or more of the settings. Pressing on control 82p will graphically display one or more user-selected parameters on display 88, such as, but not limited to, the measured and recorded patient temperatures, the target temperature, the fluid temperature and working capacity. A help control 82q, when pressed, causes controller 60 to display contextual help screens for therapies, navigation, and button usage on display 88.

Although not illustrated in FIG. 4, control panel 76 may further includes several additional controls and/or indicia. For example, in some embodiments, control panel 76 may include port icons that correspond to outlet ports 58 and that indicate, based on their illumination state (color, on/off, etc.), whether each respective port 58 is active, inactive, blocked, and/or in another state. Further details regarding one manner in which such port icons may be displayed, as well as the information conveyed by such icons, may be found in commonly assigned U.S. Pat. No. 10,390,992 issued to Hopper et al. on Aug. 27, 2019, and entitled THERMAL CONTROL SYSTEM, the complete disclosure of which is incorporated herein by reference. Thermal control unit 22 may also include any features and/or functions of the thermal control units disclosed in the aforementioned '992 patent.

Among other functions, controls 82 and/or touchscreen display 88 of control panel 76 allow a user to perform one or more of the following functions: activate/deactivate one or more of a plurality of alarms; choose the characteristics of each of the available alarms; select a thermal profile or thermal sequence for implementing the thermal therapy; define the characteristics of the selected thermal therapy; instruct thermal control unit 22 to display graph information about a thermal therapy session; select what information is included within the graph information; define characteristics of the graph information; control what information is received from any off-board sensors that are adapted to communicate with one of the transceivers 90; control what information is recorded, displayed, and/or transferred to other devices during a thermal therapy session; communicate with EMR server 124 and remote computing device 126; receive information about a patient undergoing thermal therapy; start, stop, and pause a thermal therapy session; analyze outputs from one or more sensors to determine if the patient is shivering; and other functions.

Display 88 of control panel 76 (FIG. 4) is configured to display a plurality of different screens thereon. As noted, display 88 may be a touchscreen-type display, although it will be understood that a non-touchscreen display may alternatively be used. Display 88 displays one or more visual indicators, one or more controls, and/or one or more control screens, and/or other types of information, as will be discussed more below. Display 88 may comprise an LED display, an OLED display, or another type of display. Display 88 may be configured to have its brightness level adjusted. That is, the amount of light emitted from display 88 can be varied by a controller included within thermal control unit 22. In some embodiments, the brightness is adjusted based on one or more ambient light sensors, such as is disclosed in commonly assigned U.S. patent application Ser. No. 63/31,973 filed May 29, 2020, by inventors Frank Lee et al. and entitled PATIENT SUPPORT APPARATUS WITH AUTOMATIC DISPLAY CONTROL, the complete disclosure of which is incorporated herein by reference. Still other types of brightness control, sensors, and/or sensing systems may be used.

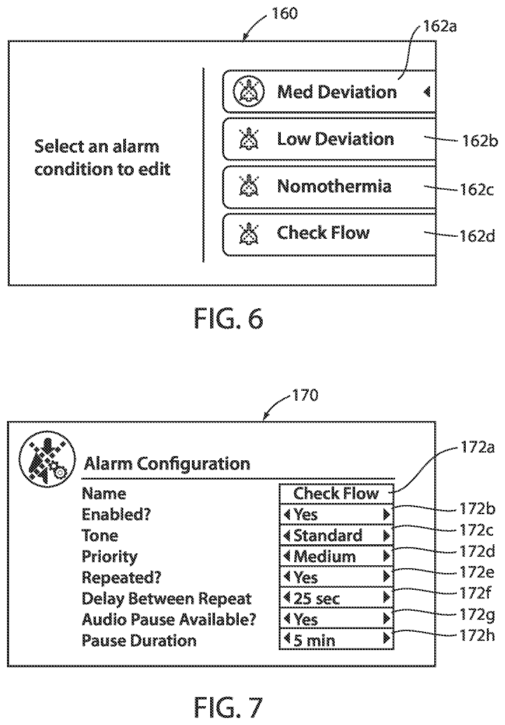

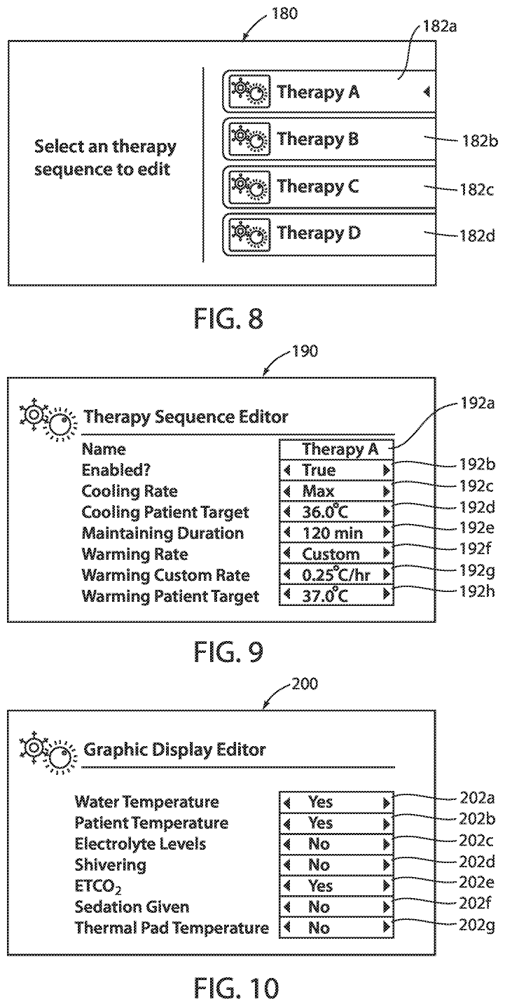

In addition to the previously described controls 82a-q, control panel 76 includes, in the embodiment shown in FIG. 4, an alarms control 82r, a therapy control 82s, a location control 82t, and a user control 82u. These controls 82r-s, when pressed, cause the display 88 to display different screens on display 88. More specifically, when a user presses alarms control 82r, control panel 76 displays an alarm selection screen on display 88, such as that shown in FIG. 6, which enables the user to select one or more particular alarms. When a user presses therapy control 82s, control panel 76 displays a therapy selection screen on display 88, such as that shown in FIG. 8, which enables the user to select one or more particular therapy profiles or sequences. When a user presses location control 82t, control panel 76 displays a location selection screen on display 88 that enables the user to select and/or confirm one or more locations within the healthcare facility in which the thermal control unit 22 is currently positioned. And when a user presses user control 82u, control panel 76 displays a user selection screen on display 88 that enables the user to select and/or confirm one or more users that are going to utilize thermal control unit 22. In some embodiments, controller 60 may also—in addition to displaying the aforementioned screens-automatically commence a function associated with those screens in response to the user pressing on the corresponding control (e.g. when a user presses on the therapy control 82s, controller 60 also automatically start implementing a particular therapy).

For all of the controls 82r-u (FIG. 4), screens other than the ones specifically mentioned above may be displayed on display 88 in other embodiments of thermal control unit 22 in response to a user pressing these controls. Thus, it will be understood that the specific screens mentioned above are merely representative of the types of screens that are displayable on display 88 in response to a user pressing on one or more of controls 82r-u. It will also be understood that, although controls 82r-u have all been illustrated in the accompanying drawings as dedicated controls that are positioned adjacent display 88, any one or more of these controls 82r-u could alternatively be touchscreen controls that are displayed at one or more locations on display 88. Still further, although controls 82r-u have been shown herein as buttons, it will be understood that any of controls 82r-u could also, or alternatively, be switches, dials, or other types of non-button controls.

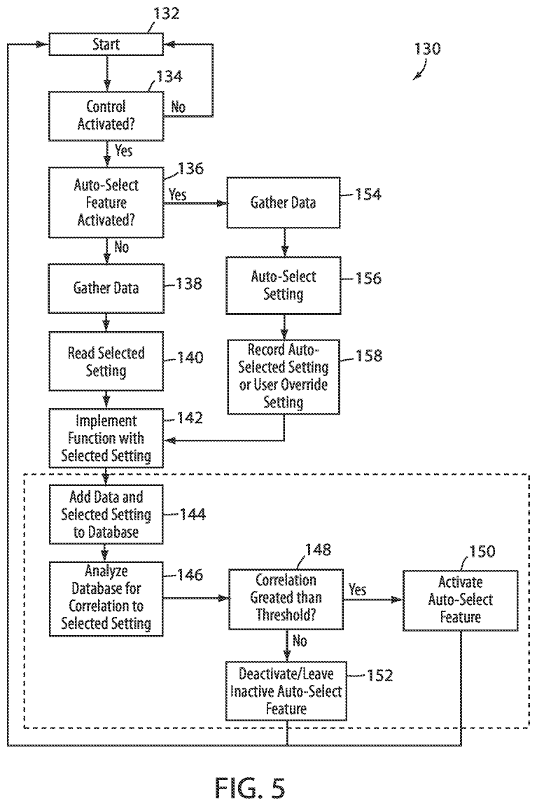

In some embodiments of thermal control unit 22, controller 60 is adapted to perform an automatic setting selection algorithm 130 in response to a caregiver or other user activating one or more controls on patient support apparatus. FIG. 5 illustrates in greater detail one embodiment of automatic setting selection algorithm 130. Algorithm 130 begins at a step 132 where it proceeds to step 134. At step 134, controller 60 determines if any control 82 that is to be monitored as part of algorithm 130 has been activated by the user. The particular controls 82 that are monitored at step 134 may vary from thermal control unit 22 and include, but are not necessarily limited to, any of the controls 82a-u discussed above. In general, the controls 82 that may be monitored at step 134 are any controls that perform any function on thermal control unit 22 wherein the function involves at least two different settings that the user can choose from. Depending upon which setting the user chooses, the function is carried out in a different manner. For example, any one or more of controls 82 that may be monitored as part of step 134 are the charting control 82p, the alarms control 82r, the therapy control 82s, the location control 82t, and/or the user control 82u. Still other controls 82 may, of course, be monitored at step 134.

If the user activates a monitored control 82 at step 134, controller 60 proceeds to step 136 (FIG. 5). If the user does not activate a control at step 134, controller 60 proceeds back to step 132, and continues to wait until a control is activated at step 134. At step 136, controller 60 determines if an auto-select feature has been activated or not. The auto-select feature is described in more detail below. In general, the auto-select feature is activated when sufficient data has been gathered from past activations of a particular control 82 at step 134 to enable controller 60 to accurately predict which setting the user will select when carrying out the function associated with that particular control 82. As will be discussed in greater detail below, this previously gathered data may be stored onboard thermal control unit 22 (e.g. in memory 80), or it may be stored at a remote computer (e.g. remote computing device 126), or it may be stored in multiple locations.

If the auto-select feature has not been activated when controller 60 performs step 134, controller 60 proceeds to step 138 (FIG. 5). At step 138, controller 60 takes readings from a plurality of sensors, including, but not limited to, any one or more of sensors discussed above that are positioned onboard thermal control unit 22. In addition to taking readings from a plurality of sensors onboard thermal control unit 22, controller 60 may gather additional data from additional sensors and/or data storage devices that are positioned off-board thermal control unit 22. Such additional data may be gathered from any of the off-board sensors and/or devices discussed above. For example, controller 60 may determine at step 138 the location of thermal control unit 22 within the healthcare facility (e.g. room number, bay identifier, ward, and/or treatment unit). Such location data may be gathered in a variety of different manners. For example, controller 60 may utilize location sensor 92 and/or it may utilize transceiver 90 to communicate with one or more servers on local area network 122 that include the current location of thermal control unit 22. Still other manners of determining the location of thermal control unit 22 may be used. In some embodiments, any of the manners of determining the location of a patient support apparatus that are disclosed in commonly assigned U.S. patent application Ser. No. 62/868,947 filed Jun. 20, 2019, by inventors Thomas Durlach et al. and entitled CAREGVER ASSISTANCE SYSTEM, may be used to determine the location of thermal control unit 22. The entire disclosure of the aforementioned '947 application is incorporated herein by reference. Still other types of location determination techniques may be used.

In addition to gathering location data, controller 60 may also and/or alternatively gather data about the patient being treated with thermal control unit 22 at step 138 (FIG. 4). Such patient data may include data indicating any of the above-mentioned patient data (e.g. height, weight, age, BMI, medication history, etc.). This data may be gathered from sending an inquiry to EMR server 124, querying data stored in memory 80, and/or in other manners. In some embodiments, some or all of this data may be gathered through communication of thermal control unit 22 with a caregiver assistance server/application of the type disclosed in the commonly assigned 62/868,947 application filed Jun. 20, 2019, and previously incorporated herein by reference.

In addition to patient data, data regarding the caregiver associated with the patient assigned to thermal control unit 22 may also be gathered at step 138. Such caregiver data may include an identification of the caregiver using thermal control unit 22 and/or other information about the caregiver (e.g. age, gender, number of years of experience, etc.). Such caregiver information may be retrieved in any of the aforementioned manners, such as by communicating with one or more servers on network 122, such as, but not limited to, a caregiver scheduling server that identifies which caregivers are assigned to which patients (or rooms or patient support apparatuses 116) and what shifts (e.g. times) the caregivers are scheduled to be present within the healthcare facility. Still other data may be gathered at step 138 beyond the data specifically mentioned above.

After gathering data at step 138, controller 60 moves to step 140 where it determines what setting the user has selected for carrying out the function associated with the control that was activated at step 134. For example, if the control activated at step 134 is an alarm activation control (e.g. control 82r or the activation of alarm characteristic 172b to an enabled state, as discussed more below), the caregiver has a choice of what alarms to activate, as well as what characteristics the alarm should have when activated. The alarm selection screen 160 and the alarm customization screen 170 of FIGS. 6 and 7, respectively, better illustrates this setting selection process. In some embodiments, controller 60 is configured to display alarm selection screen 160 in response to a caregiver pressing alarm control 82r (and/or in response to other controls 82 that may be activated). Alarm selection screen 160 includes multiple alarms 162a, 162b, 162c, etc. that the user may select. Once the user selects a particular alarm 162, controller 60 is configured to display an alarm customization screen, such as alarm customization screen 170 of FIG. 7, that displays characteristics of the selected alarm 162 and that allows the user to customize those alarm characteristics, as will be discussed in more detail below.

Each of the alarms 162 shown in FIG. 6 defines when controller 60 will issue an alarm. Although FIG. 6 only shows four such alarms 162a-d, it will be understood that controller 60 is configured to issue more than just these four alarms. In at least one embodiment, memory 80 includes a default set of alarms 162 that instruct controller 60 to issue an alarm when any one of the following conditions occur: (a) one or more of the patient temperature sensors 86 malfunctions; (b) one or more of the patient temperature sensors 86 is disconnected from its corresponding port 84; (c) the patient's temperature deviates outside of a first range (e.g. a narrow range); (d) the patient's temperature deviates outside of a second range (e.g. a wider range than the first range); (e) the patient's temperature devices from the normal human body temperature (37° C.) by more than a threshold; (f) the temperature of the fluid delivered to the outlet ports 58 deviates outside of an acceptable range; (g) a sensor (not shown) detects that there is insufficient fluid inside thermal control unit 22; (h) a flow sensor (not shown) detects that less than an acceptable amount of fluid is being pumped through or out of thermal control unit 22 (e.g. out of outlet manifold 54); (i) a user pauses a therapy session (via control panel 76); and (j) a battery included within thermal control unit 22 discharges below a threshold level. In such embodiments, controller 60 is configured to display all of these alarms 162 on alarm selection screen 160 (or alternatively it is configured to display multiple alarm selection screens 160 that collectively include all of these alarms 162).

Controller 60 monitors the conditions associated with each of the alarms 162 during operation of thermal control unit 22 and issues a corresponding alarm if it detects the occurrence of the alarm condition. Thus, for example, controller 60 monitors signals from patient temperature sensor 86 during operation of thermal control unit 22, and if those signals go outside of an expected range, or otherwise behave in a manner that is not expected, it concludes that the patient temperature sensor 86 is malfunctioning, and therefore issues an alarm corresponding to this condition. Similarly, controller 60 monitors one or more sensors (not shown) that detect the connection/disconnection of patient temperature sensor 86 to patient temperature probe port 84 and, if the sensor 86 is unplugged from the port 84, it issues the alarm corresponding to this condition. It can thus be seen that controller 60 monitors all of the corresponding conditions for alarms 162 during operation of thermal control unit 22 and issues an alarm if it detects the presence and/or occurrence of one or more of the underlying conditions.

Controller 60 is also configured to allow a user to customize the characteristics of any of the alarms 162, as well as to turn on and off these alarms 162. One manner in which controller 60 is configured to allow a user to make these types of modifications is via alarm customization screen 170 (FIG. 7). Controller 60 is configured to display alarm customization screen 170 in response to a user touching (or otherwise selecting) one of alarms 162 shown in alarm selection screen 160 (FIG. 6). In the particular example illustrated in FIGS. 6 and 7, the user has touched check flow alarm 162d in FIG. 6 and controller 60 has displayed alarm customization screen 170 in FIG. 7 that corresponds to the check flow alarm 162d. If the user were to select medium deviation condition alarm 162a from screen 160, controller 60 is configured to display an alarm customization screen 170 that is specific to the medium deviation alarm 162a. Likewise, if the user were to select low deviation alarm 162b from screen 160, controller 60 is configured to display an alarm customization screen 170 that is specific to the low deviation alarm 162b. Similarly, if the user were to select normothermia alarm 162c, controller 60 is configured to display an alarm customization screen 170 that is specific to the normothermia alarm 162c. Finally, if alarm selection screen 160 were to include additional, or different, alarms 162, controller 60 is configured to display corresponding alarm customization screens 170 that are specific to each individual alarm 162.

Each alarm customization screen 170 that controller 60 is configured to display includes a list of characteristics for the corresponding alarm 162. For example, as shown in FIG. 7, alarm customization screen 170 includes eight alarm characteristics 172a-h. It will be appreciated that not only may this number of characteristics 172 be varied, but that the specific content of any one or more of these characteristics may also or alternatively be varied. In the example shown in FIG. 7, controller 60 displays the following eight characteristics of the check flow alarm 162d: (1) the name 172a of the alarm condition; (2) the enablement/disablement state 172b of the alarm condition; (3) the tone 172c of the alarm that is issued in response to detecting the alarm condition; (4) the priority 172d of the alarm; (5) the repeat status 172e of the alarm; (6) a delay amount 172f between repetitions of the alarm; (7) an audio pause availability status 172g of the alarm; and (8) a pause duration 172h.

In one embodiment, controller 60 is configured to list these same alarm characteristics 172 on each of the customization screens 170 corresponding to each one of the alarms 162. In other embodiments, individual alarms 162 may have different sets of characteristics 172 associated with them. Regardless of the specific number of alarm characteristics 172 shown on a customization screen 170, or the specific choice of alarm characteristics 172 that are displayed on a customization screen 170, controller 60 is configured to allow a user to modify each of the alarm characteristics 172. Such modification takes place by touching, or otherwise selecting, the alarm characteristic 172 that is desired to be changed.

For example, if the user wishes to change the name of an alarm 162, he or she touches the alarm name characteristic 172a on screen 170 (FIG. 7). In one embodiment, when alarm name characteristic 172a is touched, controller 60 is configured to display an alphanumeric keyboard popup on display 88 that allows the user to type in a different name for the alarm condition. Once entered, controller 60 ceases to display the keyboard popup and controller 60 saves the new name entered by the user. Such a name change will affect the name displayed by controller 60 on alarm selection screen 160 for the corresponding alarm 162. The user is able to change any of the other alarm characteristics 172 in a similar manner; that is, by touching the characteristic 172 desired to be changed and then using the arrows positioned adjacent that characteristic 172 to change the value or setting for that particular characteristic 172.

If the user wishes to disable a particular alarm 162, he or she touches one of the arrows adjacent the “enabled” alarm characteristic 172b until the word “no” is displayed. As a result of disabling the alarm 162, controller 60 does not issue an alarm when that corresponding condition is detected. Thus, in the example of FIG. 7, if the check flow alarm 162d were disabled, controller 60 would not issue an alarm if the flow rate of the fluid within circulation channel 36 (and/or delivered to thermal pads 24) fell below the threshold that is monitored by controller 60 and otherwise used to trigger this alarm.

If the user wishes to change the tone of the sound emitted by thermal control unit 22 (such by a speaker, a beeper, a buzzer, or other sound-generating device incorporated therein), he or she touches one of the arrows adjacent the “tone” alarm characteristic 172c (FIG. 7). Touching these arrows causes controller 60 to scroll through the different options for the tone that is emitted when this alarm 162 is detected. The particular options for the “tone” characteristic may vary from thermal control unit to thermal control unit, but generally include options for at least one of the pitch, strength, quality, and/or timbre of the emitted alarm sound.

Controller 60 also enables the user to change the priority of the alarm issued for each alarm 162. To make such a change, the user touches one of the arrows adjacent the “priority” characteristic 172d (FIG. 7). Touching these arrows causes controller 60 to scroll through the different options for the priority, such as, but not limited to, a “high,” “medium,” and “low” priority. In one embodiment, controller 60 is configured to respond to a change in the “priority” characteristic 172d by changing the alarm in the manner set forth in the International Electrotechnical Commission (IEC) 60601-1-8 standard (“Audible Alarms in Medical Equipment”). In other embodiments, controller 60 may adjust the alarm priority in accordance with other standards and/or in other manners.

Controller 60 is further configured to allow the user to change whether any of the alarms issued for any of alarm conditions 102 are repeated or not. To make such a change, the user selects one of the arrows positioned adjacent the “repeated” alarm characteristic 172e (FIG. 7). Touching one of these arrows causes controller 60 to toggle between displaying a “yes” and a “no.” By selecting “no,” controller 60 will not repeat the corresponding alarm, but instead will issue it only once in response to detecting the corresponding alarm 162.

If the user chooses to have an alarm repeated, controller 60 allows the user to select how much time controller 60 waits between repetitions of the alarm. The user makes this choice by selecting one of the arrows positioned adjacent the “delay between repeat” alarm characteristic 172f. Touching the adjacent left arrow reduces the time period, while touching the adjacent right arrow increase the time period. Once the desired time period is selected, controller 60 uses the selected value as the delay period between repeated issuance of that particular alarm.

Controller 60 is also configured to allow the user to change whether any of the alarms 162 can be paused by a user. In one embodiment, when an alarm is issued, controller 60 displays a pause control (not shown) on display 88 that, when touched by a user, temporarily pauses the emitted alarm sound. In another embodiment, control panel 76 includes a dedicated control 82e (FIG. 4) that, when pressed or otherwise activated, temporarily pauses the emitted alarm sound. Regardless of the specific manner in which the pause control is implemented, if the user does not wish to be able to pause a particular alarm, he or she can disable the ability of the user to pause an alarm by changing the “audio pause available” characteristic 172g (FIG. 7). Pressing on one of the arrows adjacent to this characteristic causes controller 60 to toggle between displaying a “yes” and a “no.” When the “no” is selected, controller 60 does not allow a user to pause that particular alarm. Consequently, in those embodiments in which a pause icon is displayed on display 88, controller 60 either does not display the pause icon when the corresponding alarm is issued, or it disables the pause icon when the corresponding alarm is issued. In those embodiments in which the pause control is a dedicated control 82, controller disables that control 82 for the corresponding alarm.

If the user chooses to allow a particular alarm to be paused, controller 60 is configured to also allow the user to customize how long the alert is paused for. The user selects this pause time by touching one of the arrows positioned adjacent the “pause duration” characteristic 172h (FIG. 7). Controller 60 responds to the touching of these arrows by either decreasing the pause time (e.g. left arrow) or increasing the pause time (e.g. right arrow). Once the desired pause time value is selected, controller 60 thereafter uses the selected time value when pausing the corresponding alarm. That is, when the user presses the pause control (e.g. control 82e), controller 60 stops the audible portion of the alarm for the length of time specified by characteristic 172h, and upon expiration of that time period, resumes the audible portion of the alarm (if the condition triggering the alarm has not yet been remedied).

As was noted, the particular alarm characteristics 172 shown in FIG. 7 are but one example of the types of alarm characteristics that may be customizable by a user of thermal control unit 22. In other embodiments, additional or fewer alarm characteristics 172 may be customizable, and/or different characteristics from the specific characteristics 172 shown in FIG. 7 may be customizable. Some non-limiting examples of such additional alarm characteristics include the following: local/remote; alarm duration; alarm forwarding; alarm volume; and alarm definitions. The local/remote characteristics refers to whether the alarm is issued solely by thermal control unit 22 (local) or whether thermal control unit 22 transmits an alarm message via transceiver 90 to one or more off-board devices (remote), such as, but not limited to, computing device 126, EMR server 124, one or more portable electronic devices carried by caregivers (e.g. cell phones, tablets, laptops, etc.), or to still other types of devices. The alarm duration characteristic refers to how long the alarm will continue to persist. The alarm forwarding refers to whether notification of the alarm will be forwarded to one or more particular caregivers via transceiver 90, as well as when such forwarding takes place, to whom it is directed, and any other characteristics of the alarm forwarding. The alarm volume characteristics refers to how loud thermal control unit 22 will issue the audible portion of the alarm. The alarm definitions characteristic refers to the condition(s) that will trigger an alarm, such as, with respect to a flow rate alarm, what flow rate threshold(s) will trigger the alarm and what flow rates will not trigger the alarm. Still other characteristics may be included on screen 170 and customized by the user.

In some embodiments, the ability of a user to customize the alarms 162 and/or alarm characteristics 172 is restricted to only authorized personnel. In such embodiments, controller 60 may be configured to only allow users who enter a valid password to change the alarm settings (i.e. conditions and/or characteristics). In other embodiments, other manners of restricting access to the alarm customization features of thermal control unit 22 may be implemented, such as, but not limited to, facial recognition, fingerprint (or other biometric) recognition, etc. By restricting access to the customization features of thermal control unit 22 to only authorized personnel, the actual users of thermal control unit 22 during a therapy session may be prevented from making changes to the alarm settings. Administrators of a healthcare facility can therefore dictate what types of alarms are to be utilized, as well as their characteristics, and the nurse, doctors, and other personnel who actually use the thermal control unit 22 to treat a patient may be prevented from changing these alarm settings. It will therefore be understood that the use of the term “user” herein encompasses not only the individuals who utilize thermal control unit 22 to control a person's temperature (e.g. doctors, nurses, etc.), but also users who configure the settings of thermal control unit 22 prior to, or after, individual therapy sessions (e.g. administrators).

Returning to the auto-selection algorithm 130 of FIG. 5 and, more specifically, how it applies to the selection of alarm characteristics, controller 60 may be programmed in some embodiments to monitor the activation of control 82r at step 134. In such embodiments, the pressing of control 82r may automatically enable one or more alarms. In other embodiments, the pressing of control 82r does not enable an alarm, but instead brings up one or more screens, such as alarm selection screen 160 (FIG. 6). In those embodiments, the pressing of a particular alarm control 162 may automatically enable the corresponding alarm, in which case controller 60 monitors the pressing of an alarm control 162 at step 134 of algorithm 130. Alternatively, or additionally, controller 60 may monitor the activation of a particular alarm via the enablement of that alarm carried out through alarm characteristic 172b (FIG. 7). In such embodiments, controller 60 may be programmed to monitor the alarm characteristic 172b at step 134 and, when an alarm is enabled, proceed from step 134 to step 136. Still other variations may be implemented for what specific control, or set of controls, are monitored at step 134 of algorithm 130 when that algorithm 130 is applied to the selection of specific characteristics 172 for a particular alarm 162.

At step 140, controller 60 records the setting selected by the user which, in the example of alarm selection, corresponds to the set of characteristics 172 selected by the user via screen 170. After recording these characteristics, controller 60 proceeds to step 142 where it implements the function activated at step 134 using the setting selected by the user (or by default) at step 140. Thus, in the alarm customization example, controller 60 arms the selected alarm(s) 162 with the particular characteristics 172 that were selected by the user via screen 170 (either via active selection by the user or by leaving the defaults characteristics unchanged).

After arming the selected alarm(s) 162 with the selected characteristics 172 at step 142, controller 60 either proceeds to carry out steps 144 through 152 itself, or it transmits (via transceiver 90) a set of data to remote computing device 126 and remote computing device 126 carries out steps 144 through 152. The set of data includes the data gathered at step 138, an identification of the control activated at step 134, and an identification of the setting selected at step 140 (i.e. the set of alarm characteristics 172). At step 144, either controller 60 or remote computing device 126 adds the aforementioned set of data (gathered at steps 134, 138, and 140) to a database. The database contains similar sets of data that were gathered previously when a user activated that same control at step 134. For example, whenever control 82r (or the control for characteristic 172b, or another similar control) is activated at step 134, the data gathered at steps 134, 138 and 140 is added to the database. The database therefore contains readings of the data gathered at step 138 for each time a particular setting was selected at step 140. In other words, every time a caregiver previously chose a particular setting at step 140 (or it was chosen by default), the database contains a corresponding set of data that was gathered at step 138 for that particular setting selection.

At step 146 (FIG. 5), either controller 60 or remote computing device 126 analyzes the data in the database to determine what level of correlation exists, if any, between the data gathered at step 138 and the particular selection made at step 140. This correlation analysis may be performed in a variety of different manners, including, but not limited to, using one or more machine learning algorithms, such as, but not limited to, supervised learning methods, unsupervised learning methods, semi-supervised learning methods, reinforcement learning methods, feature learning methods, and/or self-learning methods. The model used by the machine learning algorithm may be based upon any one or more of the following: an artificial neural network, a decision tree, a support vector machine, a regression analysis, a Bayesian network, and/or a training model. Regardless of which specific machine learning algorithm and/or model is utilized, controller 60 or remote computing device 126 carry out the analysis at step 146 in order to find a reliable correlation between at least one of the items of data gathered at step 138 and the corresponding setting selected at step 140. That is, controller 60 or remote computing device 126 look for at least one item of the data (and possibly a set of such data) that, over substantially all of the past times that the control of step 134 was activated, is a reliable indicator of which setting the user will select at step 140.

Returning to the alarm characteristic example, controller 60 or remote computing device 126 is configured to look at all of the past times that a particular alarm 162 was activated and the characteristics selected for that alarm, and then see if any of the data gathered at step 138 for each of these corresponding alarm activations reliably correlates to the particular set of characteristics selected at step 140. Such analysis may determine, as an example, that a particular caregiver A always selects a set of alarm characteristics B, or that any thermal control unit 22 positioned within a particular wing of the hospital (or on a particular ward or treatment unit) has a particular set of chosen alarm characteristics 172. Alternatively or additionally, the analysis may reveal that whenever caregiver A activates check flow alarm 162d during the evening hours in a particular location of the healthcare facility, he or she chooses an alarm volume 172 that is reduced relative to an alarm volume 172 used during other times of the day, and/or at other locations. As yet another example, the analysis may reveal that if the patient is over a certain age and/or if the patient has a BMI greater than a certain threshold, the caregiver activates a remote alarm characteristic with specific alarm forwarding criteria such that he or she is reassured that any alarms issued during the thermal therapy session will be forwarded to the correct person, should the patient be left unattended at some point during the thermal therapy session. Still other types of correlations between one or more of the data items gathered at step 138 and the selection made at step 140 may be discovered as part of the analysis carried out at step 146.

After carrying out the analysis at step 146, controller 60 or remote computing device 126 compares the correlation(s), if any, determined at step 146 to a threshold at step 148 (FIG. 5). In some embodiments, the threshold is configurable by the user, such as by navigating to a setting screen, or the like, on display 88 that allows the user to change the threshold. The threshold determines how reliable the correlations need to be before controller 60 will switch to making an automatic selection in the future of which setting the user would select at step 140, thereby relieving the user of the need to make this manual selection in the future. In some instances, the threshold may be 100 percent, in which case the auto-selection feature will not be implemented unless a perfect correlation can be found between one or more of the data items gathered at step 138 and the particular setting selected at step 140. In other instances, the threshold may be less so that the auto-selection feature may be activated despite the fact that the data gathered at step 138 from past activations of the particular control (at step 134) may not allow for a completely reliable prediction to be made of which particular setting the caregiver makes at step 140.

If the correlation is determined at step 148 to be greater than the threshold (FIG. 5), controller 60 or remote computing device 126 executes step 150. At step 150, controller 60 or remote computing device 126 activates the setting auto-select feature. As noted, the setting auto-select feature is a feature in which controller 60 automatically makes the selection of a particular setting (normally performed by the user at or before step 140) in response to the user activating a particular control at step 134. Thus, for example, if the auto-select feature has been activated and the caregiver presses on a control to activate an alarm, controller 60 automatically chooses the characteristics 172 for the activated alarm so that the caregiver does not need to manually select his or her desired characteristics 172. This automatic selection is made based upon the analysis of the past history (performed at step 146) of the alarm characteristic selections that were manually made by the caregiver for past activations of that alarm.

If remote computing device 126 performs steps 144 through 152, it activates the auto-select feature at step 150 by sending a message to controller 60 (via transceiver 90) indicating that controller 60 should automatically select a setting in response to a user activating a particular control (e.g. a caregiver activating an alarm 162) on thermal control unit 22. In some embodiments, controller 60 makes this automatic selection itself, while in other embodiments, controller 60 may transmit the appropriate data to remote computing device 126 and have remote computing device 126 determine which automatic selection to make. If controller 60 performs steps 144 through 152, it activates the auto-select feature itself and does not need to transmit a message to remote computing device 126, or receive a message from remote computing device 126, in order activate the auto-select feature.

If controller 60 or remote computing device 126 determines at step 148 (FIG. 5) that the correlation is less than the threshold, it proceeds to step 152 where it deactivates the auto-select feature (if previously deactivated), or leaves the auto-select feature inactive (if previously inactive). After completing step 152 (or 150), algorithm 130 returns to start step 132.

Returning to step 136 of algorithm 130 (FIG. 5), if the auto-select feature is active at the time controller 60 executes step 136, controller 60 proceeds to step 154 instead of step 138. At step 154, controller 60 gathers data. The data that is gathered at step 154 is, in some embodiments, the same data that is gathered at step 138. In other embodiments, the data gathered at step 154 may be only that portion of the data gathered at step 138 that has been determined (via the analysis at step 146) to be reliably predictive of what setting the user will choose. In either situation, after gathering the data at step 154, controller 60 proceeds to step 156. At step 156, controller 60 uses the data gathered at step 154 along with the model previously developed via the past analyses carried out at step 146 to automatically predict what setting the caregiver will choose, and to automatically select that setting for the caregiver. In other words, controller 60 uses the machine learning algorithm of step 146 with the data gathered at step 154 to automatically select the setting for the caregiver at step 156. In the alarm customization example, controller 60 automatically selects a set of alarm characteristics 172 at step 156 in response to the caregiver activating control 82r (or in response to selecting an alarm 162 on screen 160, or in response to activating an alarm via the enablement characteristics 172b of screen 170).

In some embodiments, controller 60 carries out step 156 by plugging in the data from step 154 into the predictive model developed at step 146. In other embodiments, controller 60 sends the data gathered at step 154 (and an identification of the specific control 82 activated at step 134) to remote computing device 126 and remote computing device 126 analyzes this data to make the automatic selection of the corresponding setting. In the latter embodiment, remote computing device 126 then sends a message back to thermal control unit 22 instructing it what setting to select at step 156.

From step 156, controller 60 proceeds to step 158 where it determines whether the user will utilize the setting automatically selected at step 156 or override that automatically selected setting. Regardless of whether the user accepts the automatic setting selection or manually overrides it with a different setting, controller 60 records the selected setting (automatically made or manually overridden) and proceeds to step 142. At step 142, as described previously, controller 60 carries out the function associated with the control that was activated at step 134 using the setting that was either automatically selected at step 156 or manually overridden by the caregiver at step 158. From step 142, algorithm 130 proceeds to steps 144-152 in the manner previously described, and the setting automatically selected at step 156 (or overridden at step 158), as well as the data gathered at step 154 is added to the database at step 144 and becomes part of the corpus of data utilized by the machine learning algorithm executed by controller 60 or remote computing device 126 at step 146.

It will be understood that when remote computing device 126 is configured to carry out the analysis of step 146 (FIG. 5), it may be configured to utilize data added at step 144 from a plurality of thermal control units 22 positioned within the same healthcare facility. Thus, when a user activates a control on a particular thermal control unit 22, the data utilized in step 146 of the algorithm 130 performed for that particular thermal control unit 22 may comprise data that was gathered by remote computing device 126 from other thermal control units 22. In still other embodiments, remote computing device 126 may carry out the analysis of step 146 using data from other healthcare facilities. In such embodiments, remote computing device 126 may be positioned outside of the healthcare facility (and accessible to network 122 via the Internet), or remote computing device 126 may be in communication with another remote computer via the Internet that has access to data gathered from thermal control units 22 positioned at other healthcare facilities.

Although algorithm 130 has been primarily described so far as applying to an auto-selection feature in response to a user activating an alarm, it will of course be understood that algorithm 130 may be applied to a large number of different controls on thermal control unit 22. Several of these additional controls are described below with respect to FIGS. 8-12. Still other controls beyond those described with respect to FIG. 8-12 may be used with algorithm 130. Still further, it will be understood that a single thermal control unit 22 may use algorithm 130 for one or more different controls positioned thereon, and the auto-select feature may, at a particular time, be turned on for certain controls and turned off for certain other controls. Over time, the combination of controls for which the auto-select feature is turned on may vary. Still further, in some embodiments, thermal control unit 22 is configurable by a user as to which controls algorithm 130 is to be applied and which controls algorithm 130 is not to be applied.

FIGS. 8 and 9 illustrate a therapy selection screen 180 and a therapy customization screen 190, respectively. Together, these screens 180, 190 enable a user to select a therapy sequence to implement with thermal control unit 22, as well as to customize the therapy sequence. In some embodiments, screen 180 is displayed on display 88 in response to a user pressing on control 82s of control panel 76, although it will be understood that other triggers for displaying screen 180 may be utilized. In some embodiments, screen 190 is displayed on display 88 in response to a user selecting one of the therapies shown on screen 180, as will be discussed in greater detail below.

In the example shown in FIG. 8, therapy selection screen 180 lists four therapy profiles or sequences 182a-d (the terms “therapy profiles” and “therapy sequences” are used interchangeably herein). It will be appreciated that this number of profiles 182 may be varied. Further, although FIG. 8 identifies the four different therapy profiles 182 generically (therapy A, therapy B, etc.), in actual use, controller 60 displays a more descriptive term for the various therapies, such as, but not limited to, “cardiac arrest, “neurosurgery,” “fever,” etc. Indeed, in many embodiments, controller 60 is configured to allow the user to assign names of their choosing to the various therapy profiles 182.

Once a user selects one of the therapy profiles 182a-d displayed on therapy selection screen 180 (FIG. 8), controller 60 displays a therapy customization screen 190 that corresponds to the particular therapy profile 182 selected on therapy selection screen 180. Thus, in the example shown in FIGS. 8 and 9, the user has selected “Therapy A” on therapy selection screen 180, and controller 60 is displaying details regarding the profile for “Therapy A” on customization screen 190. These details include a plurality of therapy profile characteristics 192a-h. It will be understood that the particular number of characteristics 192, as well as their content, may be varied from the example shown in FIG. 9.

Therapy customization screen 190 (FIG. 9) includes a plurality of arrows positioned adjacent each therapy profile characteristic 192. The user touches these arrows in order to adjust each of the individual therapy profile characteristics 192 to a desired state, thereby enabling the user to customize the particular therapy profile that has been selected (e.g. the profile for Therapy A). Therapy profile characteristic 192a contains the name of the therapy profile and allows the user to assign a name to, and/or edit the name of, the corresponding therapy profile 182. Therapy profile characteristic 192b allows the user to enable usage of, or disable usage of, the corresponding therapy profile 182. Therapy profile characteristic 192c allows the user to specify what cooling rate to utilize when cooling the patient, such as, but not limited to, a low cooling rate, a medium cooling rate, and/or a maximum cooling rate.

Therapy profile characteristic 192d allows the user to specify a target temperature for the patient for the corresponding therapy profile 182. Therapy profile characteristic 192e allows the user to specify how long the patient is to be maintained at the target temperature specified by characteristic 192d. Therapy profile characteristic 192f allows the user to specify whether the warming rate of the patient after the time period specified by characteristic 192e expires will be one of the standard warming rates of thermal control unit 22, or a customized warming rate. In the example shown in FIG. 9 the user has selected a custom warming rate. Thermal profile characteristic 192g allows the user to numerically specify the actual warming rate the thermal control unit 22 will attempt to achieve during the warming phase of the thermal therapy session. Finally, thermal profile characteristic 192h allows the user to specify the temperature that the patient is to be warmed to during the warming phase of the thermal therapy session.

It will be understood that, although FIG. 9 illustrates a therapy profile that involves only a single cooling followed by a single warming, any of the therapy profiles 182 may be customized by the user to include multiple coolings and/or multiple warmings, and that the rates and target temperatures of each of these may be individually specified by the user. Still other modifications can be made to the thermal therapy profiles 182.

Thermal control unit 22 may also be configured to include multiple thermal therapy profiles 182 for the same type of therapy. The multiple therapy profiles 182 may correspond to different users of thermal control unit 22 and/or different locations of thermal control unit 22. Thus, for example, a user may create a first thermal therapy profile 182 that is used for treating a cardiac arrest patient when a first clinician is treating the patient, a second thermal therapy profile 182 that is used for treating a cardiac arrest patient when a second clinician is treating the patient, a third thermal therapy profile 182 that is used for treating a cardiac arrest patient when a third clinician is treating the patient, etc. In addition to, or in lieu of, multiple thermal therapy profiles 182 for the same treatment that differ according to the specific user, thermal control unit 22 may be customized by a user to include multiple thermal therapy profiles 182 for the same treatment that are customized according to the location of the thermal control unit 22, or that are customized according to other parameters.

Algorithm 130 (FIG. 5) may be implemented with respect to therapy customization screen 190. In such implementations, the user pressing on one or more controls-such as the therapy control 82s of FIG. 4, a selection of a particular therapy 182 of FIG. 8, or the activation of the enabled characteristic 192b of FIG. 9—may be monitored at step 134 of algorithm 130. In such embodiments, controller 60 then proceeds from step 134 to step 136 and determines if the auto-select feature has been activated or not. In this particular example, the auto-select feature refers to an automatic selection of a set of therapy characteristics 192 for a particular therapy 182. If the auto-select feature has been activated, controller 60 proceeds to steps 154, 156, and 158, where it gathers data and then automatically implements the therapy characteristics 192 deemed most likely to be chosen by the user, thereby relieving the user of having to manually set each of the individual characteristics 192, such as by pressing on the arrows next to the characteristics 192 shown in FIG. 9. As a result, when the auto-select feature is activated, the mere act of a user selecting and/or activating a particular therapy profile 182 causes controller 60 to automatically select the characteristics 192 for that particular therapy profile 182 without requiring the user to press any of the selectors associated with the characteristics 192 (unless he or she wishes to override this automatic selection).

If the auto-select feature is not activated at step 136 of algorithm 130 (FIG. 5), as applied to customization screen 190 (FIG. 9), controller 60 proceeds through steps 138-152 in the manner previously described. The data gathered at step 138 (and step 154) may be the same as, or it may be different from, the data gathered at these steps when algorithm 130 is implemented with respect to the automatic selection of one or more alarm characteristics 172, as described previously with respect to FIG. 7. Thus, controller 60 may gather data at steps 138 and 154 that relates to any one or more of the particular caregiver, location, patient, time of day, medical records, and/or from any of the sensors onboard and/or off-board thermal control unit 22, etc. and use that data in the analysis step 146 and/or in the automatic selection step 156. The result will be that, after the database has been sufficiently populated, controller 60 automatically makes the desired selections of characteristics 192 in response to a user activating a thermal therapy profile 182, thereby relieving the caregiver of having to manually make such selections.

It will be understood that the database analyzed at step 146 of algorithm 130 when algorithm 130 is applied to therapy customization will be a different database than the database used at step 146 of algorithm 130 when algorithm 130 is applied to a different automatic selection function (e.g. the automatic selection of alarm characteristics 172).

FIG. 10 illustrates a graph customization screen 200 that may be displayed on display 88 of thermal control unit 22 in response to a user pressing on graphing control 82p (or another control 82 adapted to trigger the display of customization screen 200). Graph customization screen 200 allows a user to select which conditions of thermal control unit 22 are to be graphed on display 88. In some embodiments, thermal control unit 22 includes any one or more of the graphing features disclosed in commonly assigned U.S. patent application Ser. No. 16/222,004 filed Dec. 17, 2018, by inventors Gregory S. Taylor et al. and entitled THERMAL SYSTEM WITH GRAPHICAL USER INTERFACE, the complete disclosure of which is incorporated herein by reference. In other embodiments, thermal control unit 22 includes additional and/or different graph display capabilities.

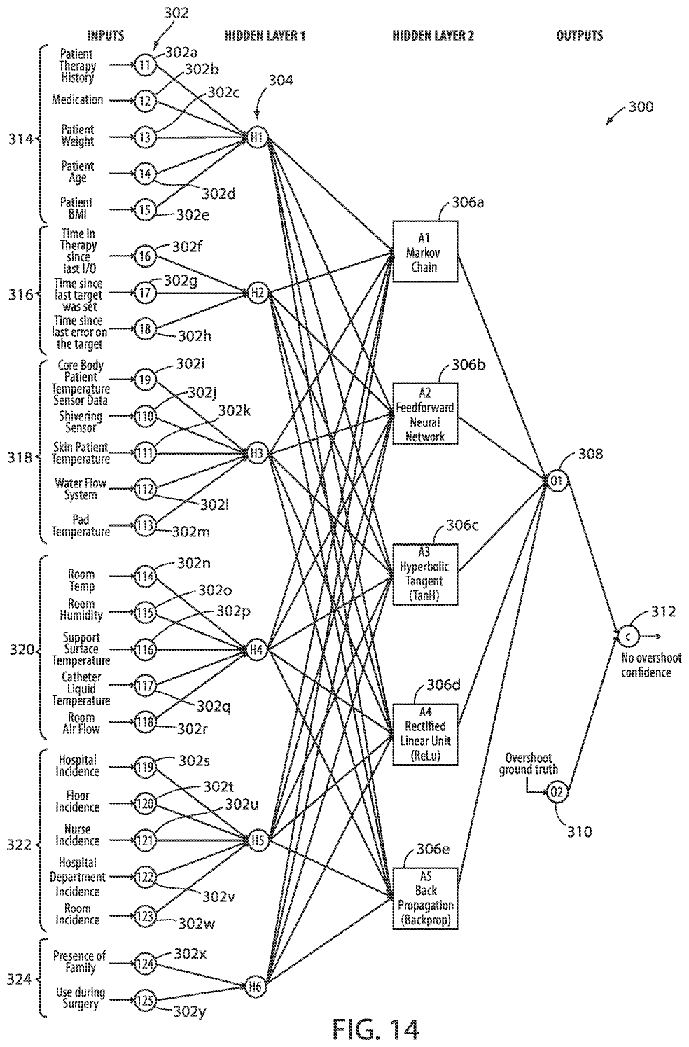

The graph customization screen 200 allows a user to customize what information is displayed in graph form on display 88. Such graph form includes a horizontal X-axis that typically is measured in units of time and one or more vertical Y-axes that plot one or more variables. One example of such a graph form is shown in FIG. 14 of the commonly assigned U.S. patent application Ser. No. 16/912,244 filed Jun. 25, 2020, by inventors Gregory S. Taylor et al. and entitled THERMAL SYSTEM WITH USER INTERFACE CUSTOMIZATION, the complete disclosure of which is incorporated herein by reference. In that example, the X-axis displays units of time and two Y-axes display units of temperature and patient potassium levels. Graph customization screen 200 allows a user to choose what parameters are shown on a graph of this type, as well as other characteristics of the graph (e.g. units of measure, color, line thickness, events, size, etc.).

Turning specifically to the content of FIG. 10, graph customization screen 200 includes a plurality of graph characteristics 202a-g. It will be understood that the specific set of graph characteristics 202a-g shown in FIG. 10 is but an illustrative example of the types of characteristics that thermal control unit 22 may be configured to allow a user to customize with respect to its graph function, and that additional and/or different graph characteristics 202 besides the ones shown in FIG. 10 may be implemented with thermal control unit 22.

A water temperature characteristics 202a allows the user to selectively display the temperature of the water (or other fluid) that is delivered to thermal pads 24. When characteristic 202a is “yes,” controller 60 displays the water temperature on the graph that is shown on display 88. When characteristics 202a is “no,” controller 60 does not display the water temperature. The same is true for the remaining characteristics 202b-g shown in FIG. 10. That is, for each one, when a “yes” is selected, controller 60 displays that characteristic on the graph shown on display 88, and when a “no” is selected, controller 60 does not display that characteristics on the graph shown on display 88.

Patient temperature characteristic 202b (FIG. 10) corresponds to the temperature of the patient as measured by the patient temperature sensor 86. Patient temperature sensors 86 measures the core temperature of the patient. In some embodiments, thermal control unit 22 also may accept readings (via auxiliary ports 94) from one or more peripheral patient temperature sensors that measure the patient's peripheral temperature. In such embodiments, graph customization screen 200 may include one or more additional characteristics 202 that allow the user to selectively display these peripheral temperature readings.

Electrolyte levels characteristics 202c corresponds to the electrolyte levels of the patient, such as, but not limited to, the patient's potassium levels. Such potassium levels may be measured by an appropriate sensor whose output is coupled to an auxiliary port 94 and/or in communication with transceiver 90. Further details about the monitoring and display of a patient's potassium levels during a thermal therapy session are disclosed in the previously mentioned U.S. patent application Ser. No. 16/912,244, and any of the features or functions disclosed therein regarding the display and/or measurement of a patient's potassium levels are incorporated herein by reference. In some embodiments, thermal control unit 22 uses the measured potassium levels in its algorithm for controlling the temperature of the circulating fluid, while in other embodiments, controller 60 merely displays and records the patient's potassium levels without using them to control the fluid temperature.

Shivering characteristic 202d corresponds to the detection of shivering in the patient. When “yes” is selected, controller 60 displays on the graph an indication of when the patient is shivering (and, in some cases, an indication of when the patient is not shivering). When “no” is selected, it does not display such shivering information on the graph shown on display 88. Any conventional means for detecting shivering may be used. Any one or more shivering sensors may be coupled to one or more auxiliary ports 94 and/or in communication with transceiver 90. In some embodiments, thermal control unit 22 may be configured to detect patient shivering, and/or react to patient shivering, in any of the manners disclosed in commonly assigned U.S. patent application Ser. No. 15/820,558 filed Nov. 22, 2017, by inventors Gregory S. Taylor et al. and entitled THERMAL SYSTEM, the complete disclosure of which is incorporated herein by reference.

ETCO₂ characteristic 202e corresponds to the end tidal carbon dioxide levels of the patient. Such carbon dioxide levels may be measured by an appropriate sensor whose output is coupled to an auxiliary port 94 and/or in communication with transceiver 90. Further details about the monitoring and display of a patient's ETCO₂ levels during a thermal therapy session are disclosed in the previously mentioned PCT patent application PCT/US2018/066114, and any of the features or functions disclosed therein regarding the display and/or measurement of a patient's ETCO₂ levels are incorporated herein by reference. In some embodiments, thermal control unit 22 uses the measured ETCO₂ levels in its algorithm for controlling the temperature of the circulating fluid, while in other embodiments, controller 60 merely displays and records the patient's ETCO₂ levels without using them to control the fluid temperature.

The “sedation given” characteristics 202f corresponds to the administration of a sedative, or other drug (such as, but not limited to, an anti-shivering drug) to the patient. When this characteristic is “yes,” controller 60 displays on the graph an indication of when the patient was given a sedative (and, in some instances, an identification of the specific sedative and/or its dosage). When this characteristic is “no,” controller 60 does not display on the graph an indication of when the patient was given a sedative. Examples of manners in which such sedative information may be displayed on the graph are found in the previously mentioned U.S. patent application Ser. No. 16/222,004, the complete disclosure of which is incorporated herein by reference.

The thermal pad temperature characteristic 202g corresponds to the temperature measured in one or more of the thermal pads 24. As was noted previously, in some embodiments, thermal pads 24 may be constructed to include one or more temperature sensors integrated therein that measure the temperature at the thermal pad 24. In such embodiments, characteristic 202g is displayed on customization screen 200 in order to allow the user to selectively display or not display this parameter on the graph shown on display 88.

As was noted previously, the characteristics 202 shown in FIG. 10 are but a representative example of the types of characteristics that are capable of being displayed on any one or more of the graphs shown on display 88. A non-exhaustive list of some additional characteristics that may also, or alternatively, be listed on customization screen 200 include the following: the total fluid flow rate, the individual flow rates for each thermal pad, the heat loss or heat gain delivered to the patient (a.k.a. the “heat quantity” or “Q” value), the one or more peripheral patient temperatures, the power exerted by thermal control unit 22, an estimated time to the target temperature, indications of statistical quantities from past thermal therapy sessions (e.g. the median amount of time to achieve a desired temperature), etc. Further information regarding the display of the estimated time to the target temperature and the statistical quantities from past thermal therapy sessions may be found in commonly assigned U.S. patent application Ser. No. 16/912,256 filed Jun. 25, 2020, by inventors Gregory S. Taylor et al. and entitled THERMAL SYSTEM WITH IMPROVED USER INTERFACE, the complete disclosure of which is incorporated herein by reference.

In some embodiments, algorithm 130 (FIG. 5) is implemented with respect to graph customization screen 200. In such implementations, the user pressing on one or more controls, such as control 82p (FIG. 4), or another control that brings about the display of a graph on display 88 and/or customization screen 200, is monitored at step 134 of algorithm 130. In such embodiments, controller 60 then proceeds from step 134 to step 136 and determines if the auto-select feature has been activated or not. In this particular example, the auto-select feature refers to an automatic selection of which characteristics 202 are to be displayed on the graph shown on display 88. If the auto-select feature has been activated, controller 60 proceeds to steps 154, 156, and 158, where it gathers data and then automatically activates the display of the selected characteristics 202, thereby relieving the user of having to manually activate each characteristic 202 that is desirably displayed and manually deactivate each characteristics 202 that is not desirably displayed. As a result, in some embodiments, when the auto-select feature is activated, the mere act of a user pressing on control 82p causes controller 60 to automatically begin displaying a graph that includes the auto-selected characteristics 202 that controller 60 deems to be most likely to be desired by the user, as determined from its previous analysis carried out at step 146. The user is, as noted, free to override or modify this automatic selection by pressing on any of characteristics 202a-g.

If the auto-select feature is not activated at step 136 of algorithm 130, as applied to graph customization screen 200, controller 60 proceeds through steps 138-152 in the manner previously described. The data gathered at step 138 (and step 154) may be the same as, or it may be different from, the data gathered at these steps when algorithm 130 is implemented with respect to the automatic selection of alarm characteristics 172, and/or the automatic selection of therapy sequence characteristics 192, as described previously with respect to FIGS. 6-9. Thus, controller 60 may gather data at steps 138 and 154 that relates to any one or more of the particular caregiver, location, patient, time of day, or outputs from any of previously described sensors (whether onboard thermal control unit 22 or off-board), and/or other data, and use that data in the analysis step 146 and/or in the automatic selection step 156. The result will be that, after the database has been sufficiently populated, controller 60 automatically makes the desired selections of characteristics 202 in response to a user activating the graph display function, thereby relieving the caregiver of having to make such selections.

It will be understood that the database analyzed at step 146 of algorithm 130 when algorithm 130 is applied to the graph characteristics 202 selection will be a different database than the database used at step 146 of algorithm 130 when algorithm 130 is applied to other automatic selection functions (e.g. the automatic alarm characteristic 172 selection and/or the automatic therapy sequence characteristics 192 selection).

It will also be understood that, although no graph selection screen has been illustrated herein, thermal control unit 22 may be configured to allow the user to select from different types and/or styles of graphs, and that each type or style may then include a corresponding set of graph characteristics 202. Thus, just as thermal control unit 22 includes an alarm selection screen 160 and a therapy selection screen 180, thermal control unit 22 may include a graph selection screen (not shown) that allows a user to select a type or style of graph, and then customize the selected graph using graph customization screen 200.

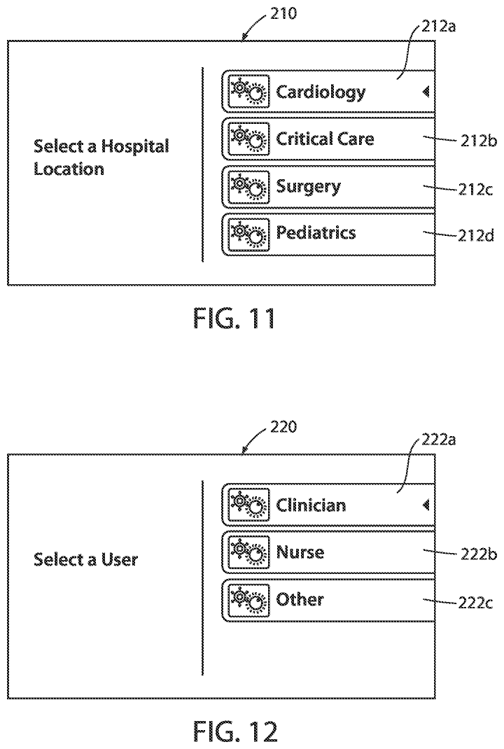

FIG. 11 illustrates a location selection screen 210 that may be displayed on display 88 of thermal control unit 22 in response to a user pressing on location control 82t. Location selection screen 210 allows a user to select different locations within a hospital (or other type of healthcare facility). Location selection screen 210 also includes a listing of locations 212a-d. Each location 212 corresponds to a particular location within the hospital, or other healthcare facility, in which thermal control unit 22 is used. Although FIG. 11 shows four specific locations 212, it will be understood that thermal control unit 22 may include more than, or less than, four locations, and that the specific locations identified in FIG. 11 may be varied. In the particular example of FIG. 11, location 212a corresponds to the cardiology department of the healthcare facility; location 212b corresponds to the critical care department of the healthcare facility; location 212c corresponds to the surgical department of the healthcare facility; and location 212d corresponds to the pediatrics department of the healthcare facility.

When a user of thermal control unit 22 selects one of locations 212a-d, controller 60 is configured, in some embodiments, to allow a user to associate one or more of the following with the selected location: a particular alarm 162 or set of alarms 162; a particular set of alarm characteristics 172; a particular therapy 182 or set of therapies 182; a particular set of therapy characteristics 192; a particular graph or set of graphs; and/or a particular set of graph characteristics 202. Still other functions or entities may also or alternatively be associated by the user in response to a location selection, including, but not limited to, one or more user customizable characteristics of those functions or entities.

Controller 60, in some embodiments, applies algorithm 130 to the selection of a location. In such embodiments, if the user presses on control 82t, or some other control that allows the user to select a location, controller 60 proceeds to step 136 of algorithm 130. In such embodiments, the “setting” referred to in algorithm 130 corresponds to the specific settings that the user has associated with a particular location (e.g. alarms, therapies, graphs, etc.). If the auto-select feature of algorithm 130 has been activated (e.g. controller 60 proceeds to steps 154-158), then controller 60 automatically selects a set of alarm(s), therapy(ies), graph(s), and/or other parameters associated with the selected location, thereby relieving the user of having to manually implement these selections.

More particularly, in those embodiments where algorithm 130 (FIG. 5) is implemented with respect to location selection, controller 60 monitors the user pressing of one or more controls that select a particular location for thermal control unit 22 and/or the outputs of one or more sensors that automatically determine a current location of thermal control unit 22. When the location of thermal control unit 22 is determined (either by manual input or automatic detection), controller 60 proceeds from step 134 to step 136 and determines if the auto-select feature has been activated or not. If the auto-select feature has been activated, controller 60 proceeds to steps 154, 156, and 158, where it gathers data and then automatically activates those settings (e.g. alarm(s), therapy(ies), graph(s)), and/or other parameters) that are associated with the current location of thermal control unit 22, thereby relieving the user of having to manually implement such settings. As a result, when the auto-select feature is activated, the mere act of a user pressing on a location selection control causes controller 60 to automatically implement settings for carrying out one or more aspects of a thermal therapy session that controller 60 deems to be most likely to be desired by the user, as determined from its previous analyses carried out at step 146. The user is, as noted, free to override or modify this automatic selection by pressing on the appropriate controls as part of step 158.

If the auto-select feature is not activated at step 136 of algorithm 130, as applied to the location selection, controller 60 proceeds through steps 138-152 in the manner previously described. The data gathered at step 138 (and step 154) may be the same as, or it may be different from, the data gathered at these steps when algorithm 130 is implemented with respect to other functions. Thus, controller 60 may gather data at steps 136 and 154 that relates to any one or more of the particular caregiver, location, patient, time of day, or outputs from any of the sensors described herein, and/or other data, and use that data in the analysis step 146 and/or in the automatic selection step 156. The result will be that, after the database has been sufficiently populated, controller 60 automatically makes the desired selections of settings (e.g. alarm(s), therapy(ies), graph(s)) in response to a user selecting a location, thereby relieving the caregiver of having to make such selections.

It will be understood that the database analyzed at step 146 of algorithm 130 when algorithm 130 is applied to the location selection will be a different database than the database used at step 146 of algorithm 130 when algorithm 130 is applied to other automatic selection functions.

FIG. 12 illustrates a user selection screen 220 that may be displayed on display 88 of thermal control unit 22 in response to a user pressing on user control 82u. User selection screen 220 allows a user to select different types of users for using thermal control unit 22. User selection screen 220 includes a listing of users 222a-c. Although FIG. 12 shows three classes of users 222a-c, it will be understood that thermal control unit 22 may include more than, or less than, three classes of users, and that the specific classes identified in FIG. 12 may be varied. In the particular example of FIG. 12, user class 222a corresponds to clinicians; user class 222b corresponds to nurses; and class 222c corresponds to other types of users.

When a user of thermal control unit 22 selects one of the user types 222a-d, controller 60 is configured, in some embodiments, to allow a user to associate one or more of the following with the selected user: a particular alarm 162 or set of alarms 162; a particular set of alarm characteristics 172; a particular therapy 182 or set of therapies 182; a particular set of therapy characteristics 192; a particular graph or set of graphs; and/or a particular set of graph characteristics 202. Still other functions or entities may also or alternatively be associated by the user in response to a user type selection, including, but not limited to, one or more user customizable characteristics of those functions or entities.

Controller 60, in some embodiments, applies algorithm 130 to the selection of a user. In such embodiments, if the user presses on control 82u, or some other control that allows the user to select a user type 222, controller 60 proceeds to step 136 of algorithm 130. In such embodiments, the “setting” referred to in algorithm 130 corresponds to the specific settings that the user has associated with a particular user type 222 (e.g. alarms, therapies, graphs, etc.). If the auto-select feature of algorithm 130 has been activated (e.g. controller 60 proceeds to steps 154-158), then controller 60 automatically selects a set of alarm(s), therapy(ies), graph(s), and/or other parameters associated with the selected user types 222, thereby relieving the user of having to manually implement these selections.

More particularly, in those embodiments where algorithm 130 (FIG. 5) is implemented with respect to user selection, controller 60 monitors the user pressing of one or more controls that select a particular user type for thermal control unit 22 and/or the outputs of one or more sensors that automatically determine a current user of thermal control unit 22. When the current user of thermal control unit 22 is determined (either by manual input or automatic detection), controller 60 proceeds from step 134 to step 136 and determines if the auto-select feature has been activated or not. If the auto-select feature has been activated, controller 60 proceeds to steps 154, 156, and 158, where it gathers data and then automatically activates those settings (e.g. alarm(s), therapy(ies), graph(s)), and/or other parameters) that are associated with the current user of thermal control unit 22, thereby relieving the user of having to manually implement such settings. As a result, when the auto-select feature is activated, the mere act of a user pressing on a user selection control causes controller 60 to automatically implement settings for carrying out one or more aspects of a thermal therapy session that controller 60 deems to be most likely to be desired by that particular user, as determined from its previous analyses carried out at step 146. The user is, as noted, free to override or modify this automatic selection by pressing on the appropriate controls as part of step 158.

If the auto-select feature is not activated at step 136 of algorithm 130, as applied to the user selection, controller 60 proceeds through steps 138-152 in the manner previously described. The data gathered at step 138 (and step 154) may be the same as, or it may be different from, the data gathered at these steps when algorithm 130 is implemented with respect to other functions. Thus, controller 60 may gather data at steps 136 and 154 that relates to any one or more of the particular caregiver, location, patient, time of day, or outputs from any of the sensors described herein, and/or other data, and use that data in the analysis step 146 and/or in the automatic selection step 156. The result will be that, after the database has been sufficiently populated, controller 60 automatically makes the desired selections of settings (e.g. alarm(s), therapy(ies), graph(s)) in response to a user selecting a user type 222, thereby relieving the caregiver of having to make such selections.

It will be understood that the database analyzed at step 146 of algorithm 130 when algorithm 130 is applied to the user type selection will be a different database than the database used at step 146 of algorithm 130 when algorithm 130 is applied to other automatic selection functions. It will also be understood that thermal control unit 22 may be detected to automatically detect the user type in a variety of different manners. Several examples of such automatic user detection are disclosed in the previously mentioned U.S. patent application Ser. No. 16/912,244, the complete disclosure of which is incorporated herein by reference.

It will be understood that the database analyzed at step 146 of algorithm 130—whether algorithm 130 is applied to any of the setting selections discussed above with respect to FIGS. 6-12, or to still other setting selections—will likely include data from more sensors (or other sources) than what controller 60 deems is necessary for implementing the auto-selection feature. In other words, controller 60 will gather data at step 138 from a set of sensors (which may potentially include off-board data storage device) that likely includes more data than is needed to accurately predict which setting should be selected at step 156. This additional data is analyzed at step 146, but controller 60 may determine that it does not have sufficient predictive power to predict which setting the user prefers in response to the particular control activated at step 134. Accordingly, controller 60, after populating the database sufficiently to determine a correlation between the outputs of one or more sensors (and/or other sources) and the user's desired selection, may no longer use the outputs from all of the sensors (or other sources) read at step 138 (or 154) to predict the user's selection. Stated still more simply, controller 60 may gather data from a relatively large set of sensors (and/or other sources) at step 138, but only use the outputs of a subset of those sensors (and/or other sources) at step 156 when automatically selecting the user's preferred setting. As noted, this is because controller 60 searches for correlations between the entire set of data gathered at step 138 and the user's desired setting, but such correlations typically only exist for a subset of that data (and/or a subset of the sensors). Accordingly, the sensors (and/or other sources) whose outputs are not predictive of the user's desired setting are not used when automatically selecting the user's preferred setting at step 156.

In some embodiments, the analysis carried out at steps 144 and 146 is the development of an algorithm for predicting what setting the user will select in response to the control selected at step 134. In such embodiments, if the analysis is carried out by remote computing device 126, remote computing device 126 transmits to thermal control unit 22 a new algorithm after the database has been sufficiently populated with enough data for remote computing device 126 to conclude that the new algorithm is sufficiently reliable for predicting the user's setting selection. The transmission of the new algorithm represents the activation of the auto-selection feature such that, when controller 60 reaches step 136 and it has the new algorithm onboard, it proceeds to steps 154-158 and uses the new algorithm to automatically select one or more settings for the user.

As mentioned above, the new algorithm may not utilize all of the outputs from the sensors that is collected at step 138. That is, either controller 60 (or remote computing device 126) uses data from a relatively large set of sensors to determine if an algorithm can be formulated with sufficient predictive power (done at steps 144-148). To the extent such an algorithm can be formulated, it typically will utilize only a subset of the data analyzed at steps 144-148. Accordingly, controller 60 (or remote computing device 126) uses a broad set of information in its search for a sufficiently reliable algorithm and when that algorithm is found, it typically will only use a subset of that broad set of information when implementing the new algorithm for predicting and/or automatically selecting the user's preferred setting(s).

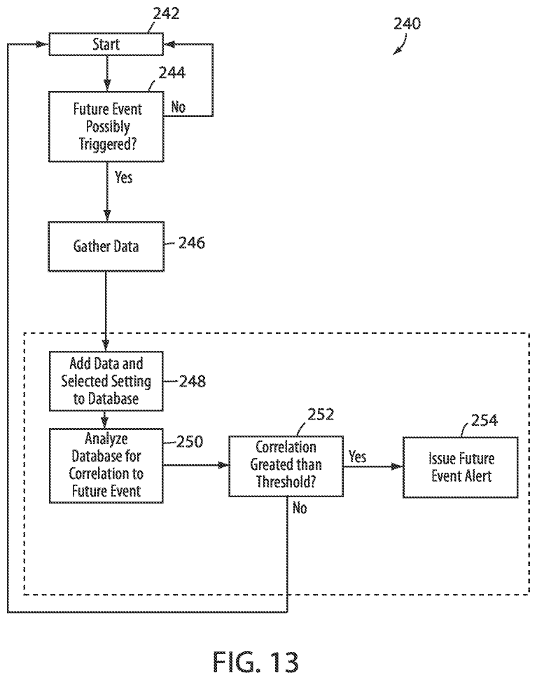

FIG. 13 illustrates a future event prediction algorithm 240 that controller 60 is adapted to execute in some embodiments of thermal control unit 22. In some of those embodiments, controller 60 is adapted to execute both algorithm 130 and algorithm 240. While in other embodiments, controller 60 may be adapted to execute only a single one of algorithms 130 or 240. In still other embodiments, controller 60 may be adapted to execute still other artificial intelligence and/or machine learning algorithms for automatically selecting one or user-preferred settings and/or predicting one or more future events that are associated with thermal control unit 22.

Algorithm 240 starts at 242 where it proceeds to step 244. At step 244, controller 60 determines if any action has taken place that will trigger it to begin monitoring for the possibility of a future event. Both the action that triggers the monitoring, as well as the future event, may vary from thermal control unit 22 to thermal control unit 22. In general, the action that triggers the commencement of monitoring for a future event includes, but is not limited to, the powering of thermal control unit 22 and/or the commencement of a thermal therapy session. The future event that is predicted by algorithm 240 may vary widely. However, for purposes of discussion herein, the future event will be focused on the commencement of shivering by the patient and/or the overshooting of the patient's temperature from the target temperature for the patient (or, conversely, the avoidance of overshoot or the avoidance of shivering). With respect to “overshoot,” this refers to how much the patient's temperature moves past the target temperature. Thus, for example, if the patient it to be cooled to thirty-five degrees Celsius, and during that cooling, the patient's temperature drops to thirty-four degrees Celsius before warming back up to thirty-five degree, there was an overshoot of one degree Celsius. In some embodiments, the triggering action may be user-customizable such that, for example, the amount of overshoot that is monitored and/or predicted by algorithm 240 is not all overshoot, but only that overshoot that exceeds a user-defined amount.

If no triggering action is detected at step 244, controller 60 proceeds back to step 242, and continues to wait until a triggering action is detected at step 244. At step 246, controller 60 takes readings from a plurality of sensors, including, but not limited to, any one or more of sensors discussed above (whether onboard thermal control unit 22 or off-board thermal control unit 22, including, but not limited to, data storage devices that contain data relating to the thermal therapy session (e.g. patient's weight, height, BMI, etc.). Controller 60 may gather data at step 246 that is the same as any of the data that controller 60 gathers at step 138 of algorithm 130, as discussed above, although it is not necessary for the data gathered at these two steps to be the same.

The information gathered at step 246 also includes an indication of a “ground truth” about the future event that algorithm 240 is being used to predict. Thus, for example, if algorithm 240 is being used to predict the patient shivering, data is gathered at step 246 indicating whether the patient has in fact started to shiver. As another example, if algorithm 240 is being used to predict patient temperature overshoot, data is gathered at step 246 indicating whether the patient's temperature has moved beyond the patient target temperature. Alternatively, if the converse of either event is being predicted (i.e. no shivering or no overshoot), data is gathered at step 246 indicating whether the patient is not shivering or whether the patient has not overshot the desired temperature. Such “ground truth” data may be gathered from one or more sensors (e.g. patient temperature sensor 86 and/or shivering detection sensors) that communicate with auxiliary ports 94 and/or transceiver(s) 90).

After gathering the data at step 246, controller 60 either processes the gathered data itself or it sends the data to an off-board computer device (e.g. remote computing device 126) for processing. That is, as with steps 144 through 152 of algorithm 130, which may be either performed by controller 60 or an off-board computer device, so to may steps 248 through 254 of algorithm 240 be performed either by controller 60 or an off-board computing device (or multiple off-board computing devices, or by a combination of controller 60 and one or more off-board computing devices).

At step 248, either controller 60 or remote computing device 126 adds the data gathered at step 246 to a database. The database contains similar sets of data that were gathered previously when the triggering action was still valid at step 244. That is, algorithm 240 repetitively loops through steps 244 through 252 until it discovers a correlation between the data in the database and the actual occurrence of the future event (the “ground truth”). The repetitive looping involves gathering data at step 246 both before the event happens and after the event is actually detected (“ground truth”). The database, which may be different from the database(s) used with algorithm 130, therefore contains readings of the data gathered at step 246 for each time the triggering action took place, as well as for each iteration through the cycle of algorithm 240 while the triggering event continued (e.g. was not cancelled), as well as one or more sets of data gathered after the event took place.

At step 250, either controller 60 or remote computing device 126 analyzes the data in the database to determine what level of correlation exists, if any, between the data gathered at step 246 and the “ground truth,” as mentioned above. This correlation analysis may be performed in a variety of different manners, including, but not limited to, using one or more machine learning algorithms, such as, but not limited to, supervised learning methods, unsupervised learning methods, semi-supervised learning methods, reinforcement learning methods, feature learning methods, and/or self-learning methods. The model used by the machine learning algorithm may be based upon any one or more of the following: an artificial neural network, a decision tree, a support vector machine, a regression analysis, a Bayesian network, and/or a training model. Regardless of which specific machine learning algorithm and/or model is utilized, controller 60 or remote computing device 126 carries out the analysis at step 250 in order to find a reliable correlation between at least one of the items of data gathered at step 246 and the “ground truth.” That is, controller 60 or remote computing device 126 look for at least one item of the data (and likely a set of such data) that, over substantially all of the past times that the triggering action of step 244 took place, is a reliable indicator of the future event taking place.

For example, if algorithm 240 is configured to predict when a patient is going to shiver (or not going to shiver), controller 60 or remote computing device 126 is configured to look at all of the data gathered at step 246 and to see if any combination of one or more of the data items correlates to the occurrence of the patient actually shivering (or not shivering). Such analysis may determine, as an example, that patients with a certain BMI level who are cooled to a certain temperature tend to start shivering, or that patients of a certain weight tend to start shivering after their ETCO2 levels reach a certain level, or after one or more of the other sensors discussed herein detect one or more conditions, and/or a sequence of conditions.

After carrying out the analysis at step 250, controller 60 or remote computing device 126 compares the correlation(s), if any, determined at step 250 to a threshold at step 252 (FIG. 13). In some embodiments, the threshold is configurable by the user, such as by navigating to a setting screen, or the like, on display 88 that allows the user to change the threshold. The threshold determines how reliable the correlations need to be before controller 60 will issue an alert of the possibility of the future event taking place (step 254). In some instances, the threshold may be 100 percent, in which case the alert will not be issued unless a perfect correlation can be found between one or more of the data items gathered at step 246 and the occurrence of the future event has been definitively established. In other instances, the threshold may be less so that an alert (or warning) of the future event taking place is issued when the probability of that event occurring is over the threshold.

If the correlation is determined at step 252 to be greater than the threshold (FIG. 13), controller 60 or remote computing device 126 executes step 254. At step 254, controller 60 or remote computing device 126 issues an alert to the user. The alert may take on different forms, including a local alert (local to thermal control unit 22) that involves any combination of audio and/or visual indications on thermal control unit 22. Still further, the alert may be a remote alert (to thermal control unit 22) in which case the alert is either transmitted off of thermal control unit 22 via transceiver 90 (or another transceiver) and communicated to one or more servers (or it is issued by one or more servers, such as remote computing device 126), and, in some embodiments, further communicated to one or more mobile electronic devices carried by one or more personnel of the healthcare facility. In general, the alert may be customizable with respect to any of the alarm characteristics 172 previously discussed. In some embodiments, thermal control unit 22 and remote computing device 126 are integrated into a caregiver assistance system and the alert is forwarded to one or more mobile electronic devices carried by one or more caregivers using the caregiver assistance system. One example of such a caregiver assistance system into which thermal control units 22, patient support apparatuses 116, and remote computing device 126 may be integrated is disclosed in commonly assigned PCT patent application serial number PCT/US2020/039587 filed Jun. 25, 2020, and entitled CAREGIVER ASSISTANCE SYSTEM, the complete disclosure of which is incorporated herein by reference (including references incorporated by reference into the aforementioned PCT/US2020/039587 application). Still other types of alerts may be issued at step 254.

If remote computing device 126 performs steps 248 through 254, it activates the alert at step 254 by sending a message to controller 60 (via transceiver 90) indicating that controller 60 should issue a local alert at step 254. If controller 60 performs steps 248 through 254, it activates the local alert and/or sends a message to remote computing device 126 to activate the alert.

If controller 60 or remote computing device 126 determines at step 252 that the correlation is less than the threshold, it returns to step 244 and continues to cycle through algorithm 240 in the manner previously described. In other words, when returning to step 244, controller 60 checks again to see that the triggering action that was analyzed in the previous iteration of step 244 is still in its triggering state (e.g. the thermal therapy session is still continuing). If it is, it continues to step 246 and proceeds in the manner previously described. If it is not, it returns to step 242 and waits until the triggering action takes place again.

It will be understood that when remote computing device 126 is configured to carry out the analysis of step 250 (FIG. 13), it may be configured to utilize data gathered at step 246 from a plurality of thermal control units 22 positioned within the same healthcare facility. Thus, when a triggering action occurs at step 244 for a particular thermal control unit 22, the data utilized in step 250 of the algorithm 240 performed for that particular thermal control unit 22 may comprise data that was gathered by remote computing device 126 from other thermal control units 22 when the same triggering action occurred for those thermal control units 22. In still other embodiments, remote computing device 126 may carry out the analysis of step 250 using data from other healthcare facilities. In such embodiments, remote computing device 126 may be positioned outside of the healthcare facility (and accessible to network 122 via the Internet), or remote computing device 126 may be in communication with another remote computer via the Internet that has access to data gathered from thermal control units 22 positioned at other healthcare facilities.

It will be understood that any thermal control unit 22 may implement more than one instance of algorithm 240 for use in predicting the occurrence of different events. When done so, the data gathered at step 246 for the different events may be different, just as the data gathered at step 138 may be different for the different controls activated at step 134 of algorithm 130. Still further, thermal control units 22 are user-customizable, in some embodiments, as to which algorithm 130 and/or 240 is to be implemented thereon, as well as to the number of instances of the algorithm 130 and/or 240 to executed thereon and as well as the content of the algorithms 130 and/or 240. In other words, the user is free to choose, for example, which setting(s) are to be subjected to algorithm 130 and/or which future events are to be subjected to algorithm 240.

It will also be understood that both algorithms 130 and 240 may be modified substantially from what is shown in FIGS. 5 and 13, respectively. As one example of such a modification, algorithm 240 may be modified such that controller 60 and/or remote computing device 126 takes one or more actions automatically in response to a correlation being determined at step 252 that is greater than the threshold. These automatic actions may be in addition to, or in lieu of, the alert issued at step 254. Such automatic actions may include, for example, automatically implementing a change in the temperature control sequence and/or algorithm that adjusts the temperature of the fluid delivered to the thermal pads such that the patient is less likely to shiver and/or less likely to overshoot the target patient temperature.

As with algorithm 130, the database analyzed at step 250 of algorithm 240 will likely include data from more sensors (and/or other sources) that than what controller 60 deems is necessary for predicting the future event. In other words, controller 60 will gather data at step 246 from a set of sensors (and/or other sources) that likely includes more data than is needed to accurately predict (or predict with a threshold-exceeding probability) the occurrence of the future event. This additional data is analyzed at step 250, but controller 60 may determine, in some embodiments, through repetitive iterations of algorithm 240 that this data does not have sufficient predictive power to predict the future event. Accordingly, in such embodiments, controller 60, after populating the database sufficiently to determine the predictive power of the data from step 246 for the occurrence of the future event, no longer uses the data from step 246 that has insufficient predictive power for the occurrence of the future event. Stated still more simply, controller 60 gathers data from a relatively large set of sensors (and/or other sources) at step 246, but only uses the outputs of a subset of those sensors (and/or other sources) at step 252 when determining whether to issue the alert at step 254. As noted, this is because controller 60 searches for correlations between the entire set of data gathered at step 246 and the future event, but such correlations typically only exist for a subset of this data.

In some embodiments, the analysis carried out at steps 248 and 250 is the development (or improvement) of an algorithm for predicting the occurrence of the future event before the future event actually happens. In such embodiments, if the analysis is carried out by remote computing device 126, remote computing device 126 transmits to thermal control unit 22 a new algorithm after the database has been sufficiently populated with enough data for remote computing device 126 to conclude that the new algorithm is sufficiently reliable for predicting the occurrence of the future event. In such embodiments, controller 60 activates the new algorithm whenever the triggering action of step 244 occurs and uses the new algorithm to determine whether or not the probability of the future event occurring has reached such a level (e.g. more than the threshold at step 252) that an alert should be issued (e.g. step 254).

As mentioned above, the new algorithm may not utilize all of the outputs from the sensors that is collected at step 246. That is, either controller 60 (or remote computing device 126) uses data from a relatively large set of sensors (and/other sources) to determine if an algorithm can be formulated with sufficient predictive power (done at steps 218-222). To the extent such an algorithm can be formulated, it typically will utilize only a subset of the data analyzed at steps 218-222. Accordingly, controller 60 (or remote computing device 126) uses a broad set of information in its search for a sufficiently reliable algorithm and when that algorithm is found, it typically will only use a subset of that broad set of information when implementing the new algorithm for predicting and/or automatically selecting the user's preferred setting(s).

FIG. 14 illustrates one example of a neural network 300 that may be utilized by controller 60 and/or remote computing device 126 when performing any of steps 144-148 of algorithm 130 and/or any of steps 248-252 of algorithm 240. In the particular example shown in FIG. 14, neural network 300 is specifically tailored for predicting when a patient's temperature may be about to overshoot a target temperature during a thermal therapy session. It will be understood, however, that neural network 300 may be modified for predicting other events, as well as for predicting any one or more of the user-preferred settings discussed above with respect to algorithm 130.

Neural network 300 includes a plurality of inputs 302, a plurality of first hidden layer nodes 304, a plurality of second hidden layer nodes 306, a first output 308, a second output 310, and a confidence score 312. The inputs 302 include a set of patient inputs 314 comprising inputs 302a-e, a set of time inputs 316 comprising inputs 302f-h, a set of thermal control unit inputs 318 comprising inputs 302i-m, a set of room inputs 320 comprising inputs 302n-r, a set of facility inputs 322 comprising inputs 302s-w, and a set of other inputs 324 comprising inputs 302x-y. It will be understood that both the sets of inputs 314-324 and the individual inputs 302 within those sets may be changed from what is shown in FIG. 14.

Each of these inputs 302 will now be described with more detail. Input 302a comprises a history of past thermal treatments (performed using a thermal control unit 22) for the particular patient undergoing thermal treatment. This history may be stored onboard thermal control unit 22 (e.g. in memory 80) and/or it may be stored on remote computing device 126. Such a history may be generated from recording the outputs from the sensors onboard thermal control unit 22, or off-board thermal control unit 22, during previous thermal treatments, including the patient's history with shivering and/or temperature overshoot during those previous treatments. Controller 60 may determine that a particular patient thermal history corresponds to a particular patient in multiple manners. In one such manner, controller 60 communicates with EMR server 124 and/or another server on network 122 to determine which patient is assigned to thermal control unit 22 and controller 60 then reads that patient's pervious thermal histories from server 124 and/or another server. In another manner, new patient information is entered into thermal control unit 22 by a caregiver whenever a new patient is assigned to thermal control unit 22 and controller 60 simply uses that entered information to retrieve that patient's thermal history from EMR server 124 (or memory 80, if stored therein). Still other manners may be used.

Input 302b refers to a patient's medication history, particular those medications that are currently in the patient's system or potentially in the patient's system. In some embodiments, this information is directly input into thermal control unit 22 by a user utilizing control panel 76. Alternatively, or additionally, this information may be read from EMR server 124.

Input 302c refers to the patient's weight. This input may also be input directly into thermal control unit 22 by a patient or read from EMR server 124. Alternatively, or additionally, thermal control unit 22 may be adapted to communicate directly with patient support apparatus 116. In such instances, if patient support apparatus 116 includes a scale system, controller 60 can obtain this weight data from the patient support apparatus 116. Patient support apparatuses 116 that include scale systems are known, and further details regarding one such suitable patient support apparatus 116 are found in commonly assigned U.S. patent application Ser. No. 16/992,515 filed Aug. 13, 2020, by inventors Kurosh Nahavandi et al. and entitled PATIENT SUPPORT APPARATUSE WITH EQUIPMENT WEIGHT LOG, the complete disclosure of which is incorporated herein by reference.

Input 302d refers to the patient's age and input 302e refers to the patient's Body Mass Index (BMI). Both of these inputs may be determined by controller 60 sending a query to EMR server 124, or another server, and/or it may be determined by having a caregiver enter this information directly into thermal control unit 22 via a control panel 50.

Input 302f refers to the amount of time since the patient last evacuated his or her bowels and/or his or her bladder, and/or the time since the patient was administered fluids (and may include the amount of administered fluids). This information may be determined by controller 60 sending a query to EMR server 124, or another server, and/or it may be determined by having a caregiver enter this information directly into thermal control unit 22 via control panel 76.

Input 302g refers to the amount of time since the patient target temperature was set. This information may be obtained from controller 60 which, via an onboard clock, measures how long the thermal therapy session has been ongoing since the patient target temperature was input (or otherwise activated). In some cases, the thermal therapy session involves multiple patient target temperatures, and input 302g refers to the time since the most recent patient target temperature was implemented.

Input 302h refers to the amount of time since an error was detected in the patient's temperature. This reference to an “error” refers to the patient's temperature deviating from the target temperature and/or an expected temperature by more than a threshold.

Input 302i refers to the patient's core body temperature, which is directly detected by patient temperature sensor(s) 86. Input 302j refers to the patient shivering, which may be detected in any of the manners previously discussed. Input 302k refers to the patient's skin or peripheral temperature, which may be measured by one or more temperature sensors in communication with auxiliary ports 94 and/or transceiver(s) 90.

Input 302l refers to the flow volume of fluid delivered to thermal pads 24, either individually for each pad 24 or an aggregate flow volume for all of the pads. Alternatively, input 302l may refer to a flow rate. Input 302m refers to the temperature sensed by one or more temperature sensors integrated into one or more of the thermal pads 24.

Input 302n refers to a current temperature of the room in which thermal control unit 22 is currently positioned and comes from one or more room temperature sensors, either onboard thermal control unit 22 or off-board. Input 302o refers to a current humidity of the room in which thermal control unit 22 is currently positioned and comes from one or more room humidity sensors, either positioned onboard thermal control unit 22 or off-board. Input 302p refers to a temperature of the surface on which the patient is currently positioned. This may be the mattress on patient support apparatus 116, or it may be another surface. In those situations where the patient is positioned onboard a patient support apparatus 116, the mattress may be an inflatable mattress in which the temperature of the inflating fluid (e.g. air) is controlled and/or measured. In such cases, the temperature of the mattress may thus be detected and reported to thermal control unit 22 through transceiver(s) 90 and/or one or the auxiliary ports 94.

Input 302q refers to the temperature of any liquid that is being supplied to the patient via a catheter, and/or any liquid that is being drained from the patient via a catheter. This information may come from one or more sensors associated with the catheter and in communication with thermal control unit 22 via transceiver 90 and/or via one of auxiliary ports 94.

Input 302r refers to the amount of air flow in the room in which thermal control unit 22 and the patient are positioned. One or more sensors for detecting this movement of air may be incorporated into thermal control unit 22 and/or positioned in the room and in communication with thermal control unit 22.

Input 302s refers to the number of times and/or frequency at which the event that algorithm 130 or 240 is being used to anticipate or predict occurs within the healthcare facility. In the particular example shown in FIG. 14, input 302s refers to the number of times and/or frequency at which a patient's temperature overshoots the target patient temperature when being treated with thermal control unit 22 in the healthcare facility in which thermal control unit 22 is positioned. This data, in at least one embodiment, is gathered by remote computing device 126. In such embodiments, every time a thermal control unit 22 detects a patient temperature overshoot, it reports that to remote computing device 126. Remote computing device 126 saves and compiles this data from all of the thermal control units 22 and uses it when executing the neural network 300 shown in FIG. 14.

Input 302t refers to the number of times and/or frequency at which the event that algorithm 130 or 240 is being used to anticipate or predict occurs on the particular floor in which thermal control unit 22 is currently positioned. In the particular example shown in FIG. 14, input 302s refers to the number of times and/or frequency at which a patient's temperature has overshot a target temperature while under the treatment of a thermal control unit 22 that was positioned on the same floor of the healthcare facility as thermal control unit 22. As with the data for input 302s, this data, in at least one embodiment, is gathered by remote computing device 126. In such embodiments, every time a thermal control unit 22 detects a patient temperature overshoot, it reports both that fact to remote computing device 126 along with its location within the healthcare facility (or it reports a location ID that can be used by remote computing device 126 to determine the location of thermal control unit 22). This location information may be gathered by thermal control units 22 in a variety of different manners, including, but not limited to, any of the location-determining methods for patient support apparatuses disclosed in commonly assigned PCT patent application serial number PCT/US2020/039587 filed Jun. 25, 2020, by inventors Thomas Durlach et al. and entitled CAREGIVER ASSISTANCE SYSTEM, the complete disclosure of which has already been incorporated herein by reference. Remote computing device 126 is therefore able to determine which thermal control units 22 are positioned on which floors of the healthcare facility and to arrange the data it receives and saves according to the different floors of the healthcare facility.

Input 302u refers to the number of times and/or frequency at which the event that algorithm 130 or 240 is being used to anticipate or predict occurs for the same caregiver who is using or overseeing thermal control unit 22. In the particular example shown in FIG. 14, input 302u refers to the number of times and/or frequency at which a patient's temperature is overshot by a patient assigned to the same caregiver. As with the data for inputs 302s and 302t, this data, in at least one embodiment, is gathered by remote computing device 126. In such embodiments, remote computing device 126 is configured to determine the caregiver assigned to each thermal control unit 22 so that every time a thermal control unit 22 reports an patient temperature overshoot, remote computing device 126 can associate that patient temperature overshoot with a particular caregiver. As was mentioned previously, this caregiver assignment information may be gathered by remote computing device 126 in a variety of different manners, including, but not limited to, any of the caregiver-determining methods used by the caregiver assistance application disclosed in commonly assigned PCT patent application serial number PCT/US2020/039587 filed Jun. 25, 2020, by inventors Thomas Durlach et al. and entitled CAREGIVER ASSISTANCE SYSTEM, the complete disclosure of which has already been incorporated herein by reference.

Input 302v refers to the number of times and/or frequency at which the event that algorithm 130 or 240 is being used to anticipate or predict occurs within the same department or ward that thermal control unit 22 is part of. In the particular example shown in FIG. 14, input 302v refers to the number of times and/or frequency at which a patient temperature overshoot occurs with a thermal control unit 22 that is positioned within the same ward or department of the healthcare facility. As with the data for inputs 302s-u, this data, in at least one embodiment, is gathered by remote computing device 126. In such embodiments, remote computing device 126 is configured to determine the department or ward assigned to each thermal control unit 22 so that every time a thermal control unit 22 reports a patient temperature overshoot, remote computing device 126 can associate that patient temperature overshoot with a particular ward or department of the healthcare facility. This information may be input directly into remote computing device 126 by an authorized representative of the healthcare facility, or it may be obtained by remote computing device 126 querying one or more servers on network 122 that have this information available.

Input 302w refers to the number of times and/or frequency at which the event that algorithm 130 or 240 is being used to anticipate or predict occurs within the same room that thermal control unit 22 is part of. In the particular example shown in FIG. 14, input 302v refers to the number of times and/or frequency at which a patient temperature overshoot occurs with a thermal control unit 22 that is positioned within the same room of the healthcare facility. This data may be gathered by remote computing device 126 in any of the same manners discussed above for inputs 302s-v.

Input 302x refers to the presence of the patient's friend or family member in the same room as the thermal control unit 22 used with the patient. In some embodiments, the friends or family members are detected by badges, tags, smart phone apps, or other devices that are adapted to automatically communicate and/or respond to interrogations from thermal control unit 22 when they are positioned within the same room as thermal control unit 22. Alternatively, or additionally, the presence of a friend or family member may be detected by an app on the friend or family member's cell phone that requests input from the friend or family member regarding their visit to the healthcare facility. In such embodiments, the app communicates with one or more servers on network 122 and remote computing device 126 retrieves this information from those one or more servers. Still other manners of detecting the patient's friend or family members in the room and/or healthcare facility may be used.

Input 302y refers to the seasonal rates at which the event that algorithm 130 or 240 is being used to anticipate or predict occurs for that thermal control unit 22. In the particular example shown in FIG. 14, input 302y refers to the rate or frequency at which a patient temperature overshoot occurs with a thermal control unit 22 during the current season, or during another slice of time. As with the data for inputs 302s, this data, in at least one embodiment, is gathered by remote computing device 126. This data may be gathered by remote computing device 126 in any of the same manners discussed above for inputs 302s-v.

After receiving all of the inputs 302, either controller 60 (or remote computing device 126) executes the computations of neural network 300 (FIG. 14). These computations include the first hidden layer 304 which, as shown, groups together the inputs according to their respective sets 314, 316, 318, 320, 322, and 324. The outputs of the first hidden layer are forwarded to the second hidden layer, which includes nodes 306a-e. Node 306a is, in the illustrated embodiments, a Markov chain node. Node 306b is a feedforward neural network node; node 306c is a hyperbolic tangent node; node 306d is a rectified linear unit node; and node 306e is a back propagation node. It will be understood that any of the nodes 306a-e may be changed from what is illustrated in FIG. 14.

For both hidden layers 304 and 306, the number of nodes, the content of the nodes, the inputs to those nodes, and the outputs of the nodes may be modified from what is shown in FIG. 14, including, but not limited to, the substitutions, additions, and/or deletions of one or more of these nodes with other nodes and/or other types of nodes not shown in FIG. 14.

The outputs from the hidden layers 304, 306, etc. are processed by controller 60 and/or remote computing device 126 to product an output 308 that is compared to the ground truth 310 in order to determine a confidence level of neural network 300. In the illustrated embodiment, the confidence level refers to an acceptable accuracy at which neural network 300 is able to predict the overshooting of a target patient temperature before the temperature is actually overshot. This confidence level 312 is used at steps 148 and/or 252. That is, at these steps controller 60 or remote computing device 126 determines if the confidence level has exceeded the threshold defined in these steps. If so, neural network 300 may be used in the future to predict a future event, such as the selection of a particular setting (e.g. algorithm 130) or the prediction of some other type of future event (algorithm 240).

As was noted, although FIG. 14 illustrates a specific neural network 300 used for carrying out a specific event prediction (patient exiting from thermal control unit 22), it will be understood that controller 60 and/or remote computing device 126 may use not only other types of neural networks at steps 146 and 250, but also other types of machine learning algorithms. These variations include, but are not limited to, a radial basis network, a deep feed forward network, a recurrent neural network, a long/short term memory network, a gated recurrent unit, an auto encoder, a variational auto encoder, a denoising auto encoder, a sparse network, a Markov chain, a Hopfield network, a Boltzmann machine, a restricting Boltzmann machine, a deep belief network, a deep convolutional network, a deconvolutional network, a deep convolutional inverse graphics network, a generative adversarial network, a liquid state machine, an extreme learning machine, an echo state network, a deep residual network, a Kohonen network, a support vector machine, and/or a neural Turing machine.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

1. A thermal control unit for controlling a patient's temperature during a thermal therapy session, the thermal control unit comprising:
a circulation channel coupled to a fluid inlet and a fluid outlet;
a pump for circulating fluid through the circulation channel from the fluid inlet to the fluid outlet;
a heat exchanger adapted to add or remove heat from the fluid circulating in the circulation channel;
a control adapted to be activated by a user of the thermal control unit; and
a controller adapted to control the heat exchanger in order to control the patient's temperature, the controller further adapted to perform a function of the thermal control unit when the control is activated by the user, the function being performed in a plurality of different manners based upon a setting selectable by the user, wherein the controller is further adapted to record over time setting data indicating the setting selected by the user when the function is performed;
a transceiver adapted to transmit the setting data to a computing device located off-board the thermal control unit, the computing device adapted to analyze the setting data and to determine a user-preferred setting when the function is performed; and
wherein the controller is further adapted to receive a message back from the computing device indicating the user-preferred setting and to automatically select the user-preferred setting when the user activates the control.

2. The thermal control unit of claim 1 wherein the function is an implementation of a temperature alert wherein the temperature alert is issued when the patient's temperature differs from a target temperature by more than a threshold, and the user-preferred setting includes at least one of the following: an audio characteristic of the temperature alert: a priority level of the temperature alert; a repetition setting of the temperature alert; a delay period between multiple temperature alerts: a pause availability of the temperature alert; a pause duration selection of the temperature alert; or a remote notification setting of the temperature alert.

3. (canceled)

4. The thermal control unit of claim 1 wherein the function is an implementation of a flow rate alert wherein the flow rate alert is issued when a rate of flow of the fluid through the circulation channel falls below a threshold, and wherein the user-preferred setting includes at least one of the following: an audio characteristic of the flow rate alert; a priority level of the flow rate alert: a repetition setting of the flow rate alert: a delay period between multiple flow rate alerts: a pause availability of the flow rate alert: a pause duration selection of the flow rate alert; or a remote notification setting of the flow rate alert.

5. (canceled)

6. The thermal control unit of claim 1 wherein the function is an implementation of a therapy profile wherein the therapy profile dictates how the thermal control unit seeks to control the patient's temperature during the thermal therapy session, and wherein the user-preferred setting includes at least one of the following: a target temperature for the patient; a duration for which the patient is to be maintained at the target temperature: a rate at which the patient's temperature is to be cooled; a rate at which the patient's temperature is to be warmed: or a target time to achieve the target temperature for the patient.

7. (canceled)

8. The thermal control unit of claim 1 further comprising a sensor, wherein the controller is further adapted to take multiple sets of readings from the sensor and record the sets of readings, each set of the multiple sets of readings including readings taken both before and after an occurrence of an event associated with the thermal control unit; wherein the controller is further adapted to transmit the sets of readings to the computing device, and wherein the controller is further adapted to receive an algorithm back from the computing device for predicting a future occurrence of the event.

9. (canceled)

10. The thermal control unit of claim 1 further comprising a plurality of sensors, wherein the controller is further adapted to take a set of readings from the plurality of sensors, record the set of readings, and use a first subset of the set of readings in an algorithm for performing the function of the thermal control unit, wherein the first subset excludes readings from at least one sensor in the plurality of sensors and the controller is further adapted to transmit the set of readings to the computing device, to receive an improved algorithm back from the computing device and to use the improved algorithm when performing the function, wherein the improved algorithm uses a second subset of the readings from the plurality of sensors, the second subset being different from the first subset.

11. (canceled)

12. The thermal control unit of claim 10 wherein the second subset of the readings does not exclude readings from the at least one sensor in the plurality of sensors.

13. The thermal control unit of claim 10 wherein the first subset includes at least one reading from a sensor not included in the second subset.

14. A thermal control system comprising:
(a) a plurality of thermal control units for controlling a patient's temperature during a thermal therapy session, each of the thermal control units comprising:
(i) a circulation channel coupled to a fluid inlet and a fluid outlet;
(ii) a pump for circulating fluid through the circulation channel from the fluid inlet to the fluid outlet;
(iii) a heat exchanger adapted to add or remove heat from the fluid circulating in the circulation channel;
(iv) a sensor; and
(v) a controller adapted to control the heat exchanger in order to control the patient's temperature, the controller further adapted to take multiple sets of readings from the sensor and record the sets of readings, each set of the multiple sets of readings including readings taken both before and after an occurrence of an event associated with the thermal control unit; and
(vi) a transceiver; and
(b) a computing device positioned remotely from the thermal control units and in communication with the transceivers of the thermal control units, the computing device adapted to receive the sets of readings from the thermal control units and to analyze the sets of readings to determine an algorithm for predicting a future occurrence of the event using future readings from the sensors of the thermal control units.

15. The thermal control system of claim 14 wherein the event is the patient shivering.

16. (canceled)

17. The thermal control system of claim 15 wherein the sensor includes an additional sensor, the controller is adapted to receive an additional set of readings from the additional sensor, record the additional set of readings, and transmit the additional set of readings to the computing device, wherein the computing device is further adapted to analyze the additional set of readings to determine the algorithm for predicting a future occurrence of the event using future readings from both the sensor and the additional sensor of the thermal control units, and wherein the additional sensor includes at least one of the following: a fluid temperature sensor adapted to detect a temperature of the circulating fluid; a flow rate sensor adapted to detect a rate of flow of fluid through the fluid outlet: a clock; or a second transceiver adapted to receive a message from a weight sensor positioned off-board the thermal control units.

18. (canceled)

19. The thermal control system of claim 15 wherein the controller is further adapted to control a temperature of the circulating fluid using a second algorithm, wherein the second algorithm is based at least partially on outputs from the sensor; wherein the sensor includes a plurality of additional sensors, the controller is adapted to receive an additional set of readings from the additional sensors, to record the additional set of readings, to transmit the additional set of readings to the computing device, to receive an improved second algorithm back from the computing device and to use the improved second algorithm when controlling the temperature of the circulating fluid; wherein the improved second algorithm uses readings from at least one of the plurality of additional sensors; and wherein the plurality of additional sensors includes at least two of the following: a fluid temperature sensor adapted to detect a temperature of the circulating fluid; a first patient temperature sensor adapted to detect a patient's core temperature: a second patient temperature sensor adapted to detect the patient's peripheral temperature: a flow rate sensor adapted to detect a rate of flow of fluid through the fluid outlet: a clock; or a second transceiver adapted to receive a message from a weight sensor positioned off-board the thermal control units.

20-22. (canceled)

23. The thermal control system of claim 19 wherein the computing device is adapted to use a neural network to generate the improved second algorithm, and wherein the computing device is adapted to use at least two of the following as inputs into the neural network: a patient weight, a patient age, a patient Body Mass Index (BMJ), a time of day, an amount of time since the patient last exited from the thermal control unit, a calendar date, or what type of medication the patient has taken.

24. (canceled)

25. The thermal control system of claim 14 wherein the controller is further adapted to perform a function when a control is activated by a user, the function being performed in a plurality of different manners based upon a setting selectable by the user, wherein the event is the selection of the setting by the user, and wherein the algorithm is adapted to predict a future setting in response to the user activating the control.

26. A thermal control system comprising:
(a) a plurality of thermal control units for controlling a patient's temperature during a thermal therapy session, each of the thermal control units comprising:
(i) a circulation channel coupled to a fluid inlet and a fluid outlet;
(ii) a pump for circulating fluid through the circulation channel from the fluid inlet to the fluid outlet;
(iii) a heat exchanger adapted to add or remove heat from the fluid circulating in the circulation channel;
(iv) a plurality of sensors; and
(v) a controller adapted to take a set of readings from the plurality of sensors and use a first subset of the set of readings in an algorithm for performing a function of the thermal control unit, the first subset excluding readings from at least one sensor in the plurality of sensors, wherein the controller is further adapted to record the set of readings; and
(vi) a transceiver; and
(b) a computing device positioned remotely from the thermal control units and in communication with the transceivers of the thermal control units, the computing device adapted to receive the sets of readings from the plurality of thermal control units and to determine an improved algorithm for use by each of the plurality of thermal control units when performing the function.

27. The thermal control system of claim 26 wherein the controller is further adapted to receive the improved algorithm back from the computing device and to use the improved algorithm when performing the function, wherein the improved algorithm uses a second subset of the readings from the plurality of sensors, the second subset being different from the first subset.

28-29. (canceled)

30. The thermal control system of claim 27 wherein the function is cooling the patient to a target temperature, the first subset includes a patient temperature sensor adapted to measure a core temperature of the patient, and the plurality of sensors further includes at least one of the following: a fluid temperature sensor adapted to detect a temperature of the circulating fluid; a second patient temperature sensor adapted to detect a patient's peripheral temperature: a flow rate sensor adapted to detect a rate of flow of fluid through the fluid outlet: a clock; or a second transceiver adapted to receive a message from a device positioned off-board the thermal control units, wherein the message includes at least one of the following data items: an age of the patient, a weight of the patient, a height of the patient, or a Body Mass Index (BMI) of the patient.

31. The thermal control system of claim 27 wherein the function is detecting shivering in the patient and the first subset includes a patient temperature sensor adapted to measure a core temperature of the patient.

32-33. (canceled)

34. The thermal control system of claim 30 wherein the computing device is adapted to use a neural network to analyze the set of readings and determine the improved algorithm.

35. The thermal control system of claim 34 wherein the computing device is adapted to use at least three of the following as inputs into the neural network: an age of the patient, a weight of the patient, a height of the patient, a Body Mass Index (BMI) of the patient, an ambient humidity reading, an ambient air temperature reading, a catheter liquid temperature, a room air flow reading, a temperature of a support surface upon which the patient is positioned, an amount of time since the thermal therapy session began, an amount of time since a patient target temperature was set, an amount of time since the current patient temperature differed from the patient target temperature by more than a threshold: a temperature of a thermal pad fluidly coupled to the fluid inlet and fluid outlet: an incidence frequency of past patient shivering events for a healthcare facility; an incidence frequency of past shivering events for a particular department of the healthcare facility; an incidence frequency of past shivering events for a particular caregiver: an incidence frequency of past shivering events for a floor of the healthcare facility: an incidence frequency of past shivering events for a particular room in which the thermal control units are each positioned in: a presence of family members visiting the patient: or whether the thermal controls units are being used for surgery or not.

36. (canceled)
